SEQUENCE LISTING (33 sequences)

SEQ ID NO: 1 - DNA, 1020 bp, Rattus norvegicus
  source 1..1020: mol_type = genomic DNA
atatggagaggtggggtgaggggtggcagagagggatcgagagaggagagaggggaaccagatgtagcagccaggaggccaaaggtacaaaaggggtgggtaaccaaaatgtctggattatataaaaaagagccagaggtcaggcccactttgatatgttaaataggcacctcagccatttatccaggtttgaaatgtaatataatttacatccccctggcttcctagagaccgttgtttagacggatgacctctgcagaatgtttgagggtgcagtcttgcatgtactccctggtgggctttcttgggcaggatctgggcaggaatgggcttgttctagtcacccactgcgtatgatggatgaacccgcttcctagtagttaggatggcactgggggaggcgagaaattagcacacgtaacgttttcttgtgttctattgttcactaagggaccccagtcaagcaagactgggccttggaagacctagagaccaccaaacctaatctctaccccgggtctgagtacacagggactcagagtcccaaagggggcagggcctccagacaggtggctcagaggtcccagtcctttggaaacatggcatcttcaggacactgggctttgcatctctggctgtgacagtcctttaagggagctactcctcagacatacaggagagatggtttggaaagtccgagatccaaagcctggttcaggctggactgggctgcaggctgctaagtgctcctctgccctggcatggctgggggtggggcattggctgtggtttctgaaaaagggcaaaaatgatgggaaaagctttgggatcctctgggaatcggagccgtggtaacagcagctgctgccattgctgcaaatgtttccttgagtgccagagtatgcccagagcccatccctgccgtacgccaggggaggggcgaggaccctcacagaggcagggaggccggccactcttaccacacagcagcctggctctcccacaaagaacagctatgca

SEQ ID NO: 2 - DNA, 975 bp, Homo sapiens
  source 1..975: mol_type = genomic DNA
gacttaccaggagctagggcatttataagcattgtgccctgaagataagttcaccccaggcagttctgaaaaggactcgtcaaacatgtcctgctgtagaataatttctaaaaatgtttttcagatcttaggagtaatataaacatgcagaaaagtagaaggaagaaaataatgacctgatacaaactaaaggtttgagtgctgtgtgaacagtctaagggaggttattgaattggggtgaaaagaatagaaagaagagcgcagataattgtaatagtaacaatagctaatatttattgaagctttcccatgctgggcacaggttcaagtgctttacaggggtcgcctcatttaattattactcatttctgtgtaggtgtgtagccggtgggctagttcaggttcttgactttgggacagaaaataatttgaaagtgagtcaaaagcaaaagcaagcaagagagttgactgcaaagccaaagtacactctagcagccggtcagaacgggccactcaaaggcaagacagctctgtctaatactgggggatctccctttatgggaaatttgcatgattattcatgaaggggtggtaaggggtgttgctattaagcatgttaggagtggtttcttaggtccacatgtgcagtggctgtacatgctagtacatccatcgcatgtcttataagcattttaaatctccacctgggggtgtgttttttactattataatgagcacaggtcagcccaaggacactaatcacgggtttctgtgcttgtacaaatgtggggatttttctcttctgctcctgcctttttgctgtagggtgttctaaccacgagctcaggatgcggtctgtgcactgttaggtggtttgttctcttcatcaatttgacaagtttcttgtttcctgtcaagggaggctctgaccacctcatctaacctgcctcaggtgcagccttattgccattttac

SEQ ID NO: 3 - DNA, 1360 bp, Rattus sp.
  source 1..1360: mol_type = genomic DNA
gagggacttgaaggcaggcttgggacaatttgagaatgaacccctaaggatgcttctgtgggccacagagactgctgagtggctgtgctttctggatacggtaccattttggaaagagaggagagtctctgtgccaggagaagtgtgactggtagtgaatgtgaggtttagtacggggcaacatctccacagcgctgtcaagcctgcctgcctgctcttcagctctttagctcggagatctaagggtgggggtaggaggggagccaccggaccaaatacaactgggacatcttggcaaacagcagcgggaagcaaaggggcagctgtgcaaatccttaggcaggcgggcgggcgggcaggcgggcgggcgggcgggcaggcgggcaggcgggcgggcaggcggccggatgagtagtgatggatagccaggcaggaggtggagagatctacactggagactttagaggcatctggtccttcctcacactgtccccactaccccgtacccctactccctaccccaagcaggacccagctgaatacaaccccttctcacacatgtgagtgagtgagttatccagcacataagaatgccaagctgaagacggatgattcactttggggaaggagagattttatagctcaggaaacaccaaggtttctgcctactagccaggcccttcaaaaggggaccaggatacccactgaaaagtttaatatgttgagctttcgtgcaggcctttgggggtttgggggggggggaattttgaattttttttttcgttttgtttttacctgtggtcacataaccagcacgaggcagctacaaggttcaggtctgacagagcccctgtgtccagcaccaacacctttggctatcagcctaaacctgtgccaccctgccaaagccagccttgcagaccaagagtccacccctacggtgcactaaagtcttccggattaggcacggactagggtcggggcacgattagaatcagacatgcagcaaggagtacttgagatactggactctactctccaaggtccagagattggagtcggggatgttcaaagtcaggagggaagaagagataaaatttaccttgacgtcaaaaggccctccaaattcccgctaattttaagggtggttctcactgctccccaccatcctcccacttccatcaatgacctcaatttaaattcaaatggtgtcatcttgctagatgctcggagttctggaagcaccgaggtgacgcaatctgtctggggcacggggcccttccacctattggctgcctggcgccccgggacccctcccaactaccgcggcgggaataagagcagctgcaggcgcttggaa

SEQ ID NO: 4 - DNA, 1837 bp, Homo sapiens
  source 1..1837: mol_type = genomic DNA
gatcaattaagggcatcttagaagttaggcgttcccgctgcctcctttgagcacggaggccaccaaccccctagggggaagagatgtagcgcgaggcaggggtgtcgtgctaagaaatttcgacgcttctggggactgaggacaaaggtgcggacacgaccccggggtacctggagttccgtgactcgcgccacggacggcacacctaggggctaatttctgctctgcctcaaagaacctcaagctagagtccttgcctccgcccacagccccgggatgccgctgctgcgctcaccgcacaggcagcgcccggaccggctgcagcagatcgcgcgctgcgcgttccaccgggagatggtggagacgctgaaaagcttctttcttgccactctggacgctgtgggcggcaagcgccttagtccctacctctgctgagctgaacgctcaggcacagtggaactgaaacccggttctgcgggatgtgagagctgttgaggtcacgcgtaattgggtgtgatggagggcgcctgttcgtgatgtgtgcaggtttgatgcaagcaggtcatcgtcgtgcgagtgtgtggatgcgaccgcccgagagactcggaggcaggcttgggacacgtttgagtgaacacctcaggatactcttctggccagtatctgttttttagtgtctgtgattcagagtgggcacatgttgggagacagtaatgggtttgggtgtgtgtaaatgagtgtgaccggaagcgagtgtgagcttgatctaggcagggaccacacagcactgtcacacctgcctgctctttagtagaggactgaagtgcgggggtgggggtacggggccggaatagaatgtctctgggacatcttggcaaacagcagccggaagcaaaggggcagctgtgcaaacggctcaggcaggtgatggatggcagggtaggaagggggaggtccagaggtctggatggaggcttccgcatctgtaccttgcaactcacccctcaggcccagcaggtcatcggccccctcctcacacatgtaatggatctgaagagtaccccgggacagtccggggagatggagattcggaaagtatccatggagatcttacagaatcccctatgcggaccaggaaactcttgtagatccctgcctatctgaggcccaggcgctgggctgtttctcacaatattccttcaagatgagattgtggtccccatttcaaagatgagtacactgagcctctgtgaagttacttgcccatgatcacacaaccaggaattgggccaactgtaattgaactcctgtctaacaaagttcttgctcccagctccgtctcttgtttcccacgagccctggccctctgtgggtaataccagctactggagtcagatttcttgggcccagaacccacccttaggggcattaacctttaaaatctcacttgggcaggggtctgggatcagagttggaagagtccctacaatcctggaccctttccgccaaatcgtgaaaccaggggtggagtggggcgagggttcaaaaccaggccggactgagaggtgaaattcaccatgacgtcaaactgccctcaaattcccgctcactttaagggcgttacttgttggtgcccccaccatcccccaccatttccatcaatgacctcaatgcaaatacaagtgggacggtcctgctggatcctccaggttctggaagcatgagggtgacgcaacccaggggcaaaggacccctccgcccattggttgctgtgcactggcggaactttcccgacccacagcggcgggaataagagcagtcgctggcgctgggaggc

SEQ ID NO: 5 - DNA, 1633 bp, Rattus sp.
  source 1..1633: mol_type = genomic DNA
tggccctgccctgccctgttcagaggcttcttggcagtgcggcccatttgtgctgtcctgcatccagtgtatcagcctagctaagtgtgagccatttccatttggggctactcttcagttccttttttaaaatagcttagccctctcccttacctctacccctagacagggtttcatgaattccaagcaggggcctcaactcacatttagccaggaatgatcttgaactgacctcctgagtgctggggttacaggtgtgatcaccttgctgctttaggaggtgctgggaacaacccagggcatcgtgtgtgttaagcacacactcccagctaagctacatccccagtccctctttctagaaaacatcattagttaaatatattcaggggagaagaggtcacaggtctggccagctgccccatcctttagtgcagggtcagctcccagaactgctctgctctgctctgcaagctggtgattctccttacctgtgattactccagatctgcctatttccaagatgccatttgaaggggaggggtctgcttcccactgtgactgggctatgggatccttgaccaccttgcttcatgatttgatacatttgttgtattcaaaaacttgaactgtaggatgccattaagagtctgtttatatttttggaatatttgtattacaattgttttaataaaggccggtttaaaaacctatgcatgagtgggggctgctttccttccccgccactggtcccacacacggtggacgctgttcttcccgtattccctttttggtcctaagattatagcacccagcagaacaaacactgggttctgatcaaggttgcaaaggttggactgcattagctcttctctggcccaggttggaaccaactccctctcccctgggtacttagcaaacatgtgcttgctgtgctattgactgctgggtatagactgaagcccttggggaattggggactcctgtgcatgctcttgatactgccaaacaggagccatgagactcagagcccagcacggtttccaggcacatagtaggaggacttgtaaagactaaacaaaagctttcctatcaccacaaatttgcccagaatcatctcgccatcctgaacatccaggcatacagtaggagagctctagccagccgcctgtcatcagcataatacattcatatctacaatatggcaaattcatatcctccctcactgaattattgatggactctgcacttttaaaaaaatcaatagaccaggggtggagctggagttaaaagaagcctttaaaagtctgctcttcttgtttttgctgttttgaataggagcagataaagctttccccgctggtttgaataagtcaagcccagggctaggtcggctgtgattggccagagctgggaaaatgtggttatgatgcaaacgcaagcaaatataacccagtgtctgtgtgctgtgtggccattgctgaaacaggcttcaggacccgaggtcgcctgcttcgggaccgctcgtcagtacttcacctgctgctttgctgggaaagggagaacagagggtcaggtaaggtctactgtgtgtgtccttgtcagctgaaggtgaggggacaggggtcacgccttc

SEQ ID NO: 6 - DNA, 1460 bp, Homo sapiens
  source 1..1460: mol_type = genomic DNA
gcagcacagggtggggcagcacaagttatcaaattaaattctcaaacactggctgctccgcaccattgccttgctttagtcctgctacagcagtagacaaagcgagtctgctttgagctttatggttttttgccaaagtccatactagatggcgcatgctctccaaacttggctttgtccatcaaggttcaagaaaacaatggtcagacatgttcctcttaacaaacagtatgtccccaaacagcaaaaatgcatacagtcctttctgggtgaatttttaaatcttacataaatccatcaaccccatcctttttcctttgcctcttgggagaaattaatctagctttacattaattatgcatgttatcagatttcaagctccttgagagcaggtattttaattctataaagcctctacgtggccttggacatgggtaggtgcttaattacccaagatgctccttgaatacagatggtacacgacctacacagacttagatctttaccacttcccccctctccccaccctgacttgctcaatcctgaaggaactggagacgtctaagtgtctgaggttcacgcttccacacagaagcttgggtctgtgtgggagggaaaaaggaagccatctgtccgcaggccagaccaggccacaccctgctagcacccagaaccctttgtcccaggcccagccctgccattttactttccttgcatctggaaagcacagggaatatagtagtgacaaaagaaggaagggttgtttgagtttaagaatagtttactctaaaaaaaaaaaaaaaaaaaaaaaaaggacaaaagccaaagagaaggtcaaagttgactgtggagaaggccttgcaagcagggaacttgggaagaattggaatgagagtgagagaaggcaactgagtttggaaatattttttctgactagcttttctttccaaatgccactgaacttagattggtttaggaagggttgtagtacatcaaagtggctagaagcacaggtttggggatcagataaggatttcattctagagtgtgatcttgtacaagttattcagcctttgcaaacctcagattcacacaatgtaagatgaagaaactcaccttctgaaaattagagataacatatgcaaagtgaatcaatacagggcttaacatatttatcacccctttggtaaataaccatgacgattaccagagctcttaagggcaatggcaggtgggaagcagaactcatgggtggtaatccccaggccagccaggctcaccatgtgcacttggacaagtccttgcccccatcattgtgaaatggtgcagggatgcaccatgagggtgtggcaggatggctgacaacagactgggaagcagctcggcagaaaaactggattgatgcccactatggcaagagatatcatctcccctcttgttctgtgatgtttcagtcctgg

SEQ ID NO: 7 - DNA, 351 bp, synthetic construct
  misc_feature 1..351: note = VAMP2 Antisense
  source 1..351: mol_type = other DNA
ttaagtgctgaagtaaactatgatgatgatgaggatgatggcgcaaatcactcccaagatgatcatcatcttgaggtttttccaccagtatttgcgcttgagcttggctgcgcttgtttcaaactgggaggcccccgcctggagtgcatctgcacggtcgtccagctccgacagcttctggtctcgctccaggaccttgtccacgttcaccctcatgatgtccaccacctcatccacctgggcctgggtctgctgcagtctcctgttactggtgaggtttggagggggtgcagggggaccaccctccccagccggggcagcagggggggccgtggcagcggtagcagacat

SEQ ID NO: 8 - DNA, 621 bp, synthetic construct
  misc_feature 1..621: note = SNAP25 Antisense
  source 1..621: mol_type = other DNA
ttaaccacttcccagcatctttgttgcacgttggttggcctcatcaattctggttttgttggaatcagccttctccatgatcctgtcgatctggcgattctgtgtatcgatctcattgcccatatccagggccatgtgacggaggttcccgatgatgccgctcacctgctctaggttttcatccatttcattttctcgggcatcatttgttaccctgcggatgaagccgccactgatggccatctgctcccgttcgtccactacacgagcaggctggctggccaccactccgtcctgattattgccccaggcttttttgtaagcatcacttgatttaagcttgttacaaggacatatgaaaaggccacagcatttccctaaatcttttaaatttttctcagcctccttcatgtcttggttgatatggttcatgccttcttcgacacgatcgagttgttctccttgttcatccaacataaccaaagtcctgataccagcatctttactctcttcaaccagttgcagcatacgacgggtgctttccagcgactcatcagccaactggtcagcccttcgctgcatctcctccagctcattgcgcatgtctgcgtcttcggccat

SEQ ID NO: 9 - DNA, 840 bp, synthetic construct
  misc_feature 1..840: note = STX Antisense
  source 1..840: mol_type = other DNA
ttaattcaggccaacggaaagtccaataatcaatgctaaaatgcccagcaacacaactactagcacaatgataattatcaatttcttccgggcctgactctggtatttcacagcttttttgctttcatctcgtgccttctccacgtggtccactgtgtgcatgacattcaactctatgttatctaacatctcaccctgattctccaccagcatggcgatgtccataaacatgtcgtgaagctccttgatgctgctctccagcctcacaatgtccttgtgtcgtccctcaatctcactgagggcttgcttggaaatctgtgagtcaatgatcccagaagtgaagatggccgggttgccactctccaacatctcctccagctcctcatcggttgtctttttgccagtaatttcgagctgccgctggattcgccctttgctgcgttctcggaagtccacttgagcttcattgtatttggtcatcacctccacaaacttccgagaaaggacagagtgctgggatttccgaatccgaaggtctgccgatgacctgacctcatcttcttcaatatgcttctccatgctcttcagtttgttccggacgttgttggcccttttcttaatctcagtgctgagctgctctaggtcatccttggtttttggctctggaatcggtgcagagagaatgatactgtagagtttcttagcctcctctacatgttctgagatcttgtcaatgttaagccgagtttcctcaatctcagaaaagaactcgtccataaaagccgtgttgtcgatagcaatctcaaccgcatcagtatcatcatcctgtgtcagttgcat

SEQ ID NO: 10 - protein, 457 aa, synthetic construct
  REGION 1..457: note = Light chain of botulinum neurotoxin A
MHHHHHHQFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALN

SEQ ID NO: 11 - DNA, 1374 bp, synthetic construct
  misc_feature 1..1374: note = Light chain of botulinum neurotoxin A
  source 1..1374: mol_type = other DNA
atgcaccaccaccaccaccaccagttcgtgaacaagcagttcaactacaaggaccccgtgaacggcgtggacatcgcctacatcaagatccccaacgtgggccagatgcagcccgtgaaggccttcaagatccacaacaagatctgggtgatccccgagagagacaccttcaccaaccccgaggagggcgacctgaaccccccccccgaggccaagcaggtgcccgtgagctactacgacagcacctacctgagcaccgacaacgagaaggacaactacctgaagggcgtgaccaagctgttcgagagaatctacagcaccgacctgggcagaatgctgctgaccagcatcgtgagaggcatccccttctggggcggcagcaccatcgacaccgagctgaaggtgatcgacaccaactgcatcaacgtgatccagcccgacggcagctacagaagcgaggagctgaacctggtgatcatcggccccagcgccgacatcatccagttcgagtgcaagagcttcggccacgaggtgctgaacctgaccagaaacggctacggcagcacccagtacatcagattcagccccgacttcaccttcggcttcgaggagagcctggaggtggacaccaaccccctgctgggcgccggcaagttcgccaccgaccccgccgtgaccctggcccacgagctgatccacgccggccacagactgtacggcatcgccatcaaccccaacagagtgttcaaggtgaacaccaacgcctactacgagatgagcggcctggaggtgagcttcgaggagctgagaaccttcggcggccacgacgccaagttcatcgacagcctgcaggagaacgagttcagactgtactactacaacaagttcaaggacatcgccagcaccctgaacaaggccaagagcatcgtgggcaccaccgccagcctgcagtacatgaagaacgtgttcaaggagaagtacctgctgagcgaggacaccagcggcaagttcagcgtggacaagctgaagttcgacaagctgtacaagatgctgaccgagatctacaccgaggacaacttcgtgaagttcttcaaggtgctgaacagaaagacctacctgaacttcgacaaggccgtgttcaagatcaacatcgtgcccaaggtgaactacaccatctacgacggcttcaacctgagaaacaccaacctggccgccaacttcaacggccagaacaccgagatcaacaacatgaacttcaccaagctgaagaacttcaccggcctgttcgagttctacaagctgctgtgcgtgagaggcatcatcaccagcaagaccaagagcctggacaagggctacaacaaggccctgaactga

SEQ ID NO: 12 - protein, 447 aa, synthetic construct
  REGION 1..447: note = Light chain of botulinum neurotoxin B
MHHHHHHPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 13 - DNA, 1344 bp, synthetic construct
  misc_feature 1..1344: note = Light chain of botulinum neurotoxin B
  source 1..1344: mol_type = other DNA
atgcaccaccatcatcaccaccctgttaccatcaataactttaactataacgatccaatagacaacaacaacatcatcatgatggagcccccctttgctagagggactggtcggtactacaaagcttttaagatcaccgatcggatttggattatccctgaacggtatacatttggctacaaacccgaagacttcaataaatcttctggtattttcaatcgagacgtgtgtgaatactatgatcccgactacctcaacactaacgataaaaagaacattttcctgcagacaatgattaagctgttcaatcggatcaagagtaaacccttgggtgaaaaacttctggagatgattatcaacggtataccttacctgggcgacaggagggtgccactcgaagagttcaatacaaacatagccagcgtgaccgtgaataagctgatcagtaacccaggcgaagttgagcggaagaagggaattttcgctaacctcatcatcttcggaccaggacctgtccttaacgagaatgagacaattgatattggaatccagaaccatttcgcatcacgcgaaggcttcgggggtatcatgcagatgaagttctgcccggagtatgtctctgtgttcaacaacgtgcaggaaaataagggagcgagcattttcaatcgcagaggctatttttccgaccccgcgctcatccttatgcacgagctgatccatgtcctgcacggactgtacggcatcaaagtcgacgatttgccaattgtgcccaacgaaaagaagttcttcatgcagtccaccgacgctatccaagcggaggagctctatacttttggcggacaggacccttctatcatcactccatctacagataagagtatatacgataaggttctccagaatttccgcggaatcgtcgaccgccttaacaaggtgctggtttgtatttccgacccaaacatcaatataaatatctataagaacaagttcaaagataaatataagttcgtggaggacagcgagggtaagtactctattgatgtggagagctttgataaactgtacaagtctctcatgttcggtttcacagagactaatatcgccgagaactataagataaaaacccgggcaagctatttctccgatagcctgccaccggttaagattaagaacctgctggacaatgaaatatataccatcgaggaaggatttaacatctccgacaaggacatggagaaagaataccggggacagaacaaggccattaataagcaggcttacgaggagattagcaaggagcacctggctgtgtacaaaatccagatgtgcaagtcagtgaagtag

SEQ ID NO: 14 - protein, 457 aa, synthetic construct
  REGION 1..457: note = Light chain of botulinum neurotoxin C1
MHHHHHHPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTL

SEQ ID NO: 15 - DNA, 1374 bp, synthetic construct
  misc_feature 1..1374: note = Light chain of botulinum neurotoxin C1
  source 1..1374: mol_type = other DNA
atgcaccaccaccaccaccaccccatcaccatcaacaacttcaactacagcgaccccgtggacaacaagaacatcctgtacctggacacccacctgaacaccctggccaacgagcccgagaaggccttcagaatcaccggcaacatctgggtgatccccgacagattcagcagaaacagcaaccccaacctgaacaagccccccagagtgaccagccccaagagcggctactacgaccccaactacctgagcaccgacagcgacaaggaccccttcctgaaggagatcatcaagctgttcaagagaatcaacagcagagagatcggcgaggagctgatctacagactgagcaccgacatccccttccccggcaacaacaacacccccatcaacaccttcgacttcgacgtggacttcaacagcgtggacgtgaagaccagacagggcaacaactgggtgaagaccggcagcatcaaccccagcgtgatcatcaccggccccagagagaacatcatcgaccccgagaccagcaccttcaagctgaccaacaacaccttcgccgcccaggagggcttcggcgccctgagcatcatcagcatcagccccagattcatgctgacctacagcaacgccaccaacgacgtgggcgagggcagattcagcaagagcgagttctgcatggaccccatcctgatcctgatgcacgagctgaaccacgccatgcacaacctgtacggcatcgccatccccaacgaccagaccatcagcagcgtgaccagcaacatcttctacagccagtacaacgtgaagctggagtacgccgagatctacgccttcggcggccccaccatcgacctgatccccaagagcgccagaaagtacttcgaggagaaggccctggactactacagaagcatcgccaagagactgaacagcatcaccaccgccaaccccagcagcttcaacaagtacatcggcgagtacaagcagaagctgatcagaaagtacagattcgtggtggagagcagcggcgaggtgaccgtgaacagaaacaagttcgtggagctgtacaacgagctgacccagatcttcaccgagttcaactacgccaagatctacaacgtgcagaacagaaagatctacctgagcaacgtgtacacccccgtgaccgccaacatcctggacgacaacgtgtacgacatccagaacggcttcaacatccccaagagcaacctgaacgtgctgttcatgggccagaacctgagcagaaaccccgccctgagaaaggtgaaccccgagaacatgctgtacctgttcaccaagttctgccacaaggccatcgacggcagaagcctgtacaacaagaccctgtga

SEQ ID NO: 16 - protein, 429 aa, synthetic construct
  REGION 1..429: note = Light chain of botulinum neurotoxin E3
MHHHHHHPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRK

SEQ ID NO: 17 - DNA, 1290 bp, synthetic construct
  misc_feature 1..1290: note = Light chain of botulinum neurotoxin E3
  source 1..1290: mol_type = other DNA
atgcaccatcaccatcaccatcccaagattaattctttcaactataatgatccagtcaacgacaggaccattctctacatcaagccgggcggctgtcaggaattctacaaatcatttaacatcatgaagaatatttggatcatacctgaaagaaatgtgattggaaccacaccccaggatttccaccccccaacaagcttgaaaaacggagacagttcctactatgaccctaattatcttcagagcgatgaggagaaagacagattcctgaagatcgtgaccaaaatcttcaaccggatcaacaataatctctccgggggtattttgctggaggagctgtcaaaggcgaatccttaccttggcaatgataacacaccggacaatcagttccacataggagatgcttcagctgtcgagattaaattcagcaacggttcccagcatattctgcttccgaatgtcattattatgggagccgagccagacctgttcgagaccaattcttccaacataagcttgaggaataattacatgccatctaaccacggttttgggtcaatcgcaattgtcacttttagtccggagtacagcttccgctttaatgacaacagcatcaatgaattcatacaggatcccgctctgacactcatgcatgagctgatccactctctgcatggcctgtatggtgctaaggggatcaccaccacatgtattatcacgcagcagcaaaatccactgattacaaacaggaaaggcataaatattgaagagttcctgacattcggcggcaatgaccttaacattatcactgtggctcagtataacgatatttatactaatctgctgaacgactataggaaaatcgcttcaaaactgagcaaggtgcaggtctcaaacccacagttgaatccatataaggatatctttcaggaaaagtatgggttggacaaggatgcttcaggcatctacagtgtcaatatcaacaaattcgacgacatcttgaagaaactgtatagctttacggagttcgatctggcgactaagttccaagtgaaatgccgggagacatacatcggtcaatataaatattttaaactgtccaaccttctcaatgatagcatctacaatatcagtgaaggatataacattaataacctgaaagtgaatttccgaggccagaacgccaatttgaacccccggattatcaagcccattacaggacgggggctggtgaagaaaattattcgcttttgtaagaatatcgtgtcagtgaagggaataagaaagtag

SEQ ID NO: 18 - protein, 442 aa, synthetic construct
  REGION 1..442: note = Light chain of botulinum neurotoxin F1
MHHHHHHPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRK

SEQ ID NO: 19 - DNA, 1329 bp, synthetic construct
  misc_feature 1..1329: note = Light chain of botulinum neurotoxin F1
  source 1..1329: mol_type = other DNA
atgcaccatcatcatcaccatcccgtggttatcaatagctttaattataacgatcccgtgaatgatgatacaattctctacatgcagattccatacgaggaaaagagcaagaagtattataaagcattcgaaataatgcggaacgtttggattattcccgagagaaacacaatcggaaccgacccgtccgattttgatccacccgcctcattggaaaacggcagtagcgcctactacgatcccaattatctcaccacagatgctgagaaggaccgctatctgaaaaccacaatcaagctctttaagagaatcaactctaatccagctggcgaagtcctgctgcaggaaattagctacgcaaagccatatctcggcaacgagcatacacctattaatgagttccatcccgtcactcggacgacctctgtgaacataaaaagctctacaaacgtgaagagctctataatactgaacctgctcgtgctgggtgctggcccagacattttcgagaatagttcctatccagttcgaaagttgatggattctgggggcgtgtacgatcctagcaatgacggatttgggagtattaatatagtcacattcagtcccgagtatgaatacaccttcaacgacatcagcggtggctacaattcatcaactgagagcttcattgccgacccagccatcagtctggcccatgagttgatccatgccctgcacggcctctatggggctagaggggttacctacaaggaaacaattaaagtcaagcaggctccactcatgatcgctgaaaagcccattcggctcgaggagtttctgacattcggcggccaggatctcaacataatcaccagtgctatgaaagagaagatctacaataaccttcttgcaaattacgaaaaaatcgcaacacggctgtcacgggtgaatagcgctccccctgagtacgacattaacgagtataaagattacttccagtggaaatacgggctggacaaaaatgccgacgggagctacacagtgaacgagaacaagtttaacgagatctacaagaaactgtactcatttaccgagattgacctggctaataagtttaaggtcaagtgcagaaatacttatttcatcaagtacggatttttgaaagtccctaatctgctggacgacgatatttatactgtgtctgaaggttttaatatcggaaaccttgccgtgaataaccgcgggcagaatataaagcttaatcctaagatcatcgattcaattcctgacaaaggcctcgtcgagaaaatagtgaaattctgcaaaagtgtgattcctagaaagtga

SEQ ID NO: 20 - protein, 457 aa, synthetic construct
  REGION 1..457: note = Light chain of tetanic neurotoxin
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTA

SEQ ID NO: 21 - DNA, 1373 bp, synthetic construct
  misc_feature 1..1373: note = Light chain of tetanic neurotoxin
  source 1..1373: mol_type = other DNA
atgcccatcaccatcaacaacttccggtacagcgaccccgtgaacaacgacaccatcatcatgatggagcccccctactgcaagggcctggacatctactacaaggccttcaagatcaccgaccggatctggatcgtgcccgagcggtacgagttcggcaccaagcccgaggacttcaacccccccagcagcctgatcgagggcgccagcgagtactacgaccccaactacctgcggaccgacagcgacaaggaccggttcctgcagaccatggtgaagctgttcaaccggatcaagaacaacgtggccggcgaggccctgctggacaagatcatcaacgccatcccctacctgggcaacagctacagcctgctggacaagttcgacaccaacagcaacagcgtgagcttcaacctgctggagcaggaccccagcggcgccaccaccaagagcgccatgctgaccaacctgatcatcttcggccccggccccgtgctgaacaagaacgaggtgcggggcatcgtgctgcgggtggacaacaagaactacttcccctgccgggacggcttcggcagcatcatgcagatggccttctgccccgagtacgtgcccaccttcgacaacgtgatcgagaacatcaccagcctgaccatcggcaagagcaagtacttccaggaccccgccctgctgctgatgcacgagctgatccacgtgctgcacggcctgtacggcatgcaggtgagcagccacgagatcatccccagcaagcaggagatctacatgcagcacacctaccccatcagcgccgaggagctgttcaccttcggcggccaggacgccaacctgatcagcatcgacatcaagaacgacctgtacgagaagaccctgaacgactacaaggccatcgccaacaagctgagccaggtgaccagctgcaacgaccccaacatcgacatcgacagctacaagcagatctaccagcagaagtaccagttcgacaaggacagcaacggccagtacatcgtgaacgaggacaagttccagatcctgtacaacagcatcatgtacggcttcaccgagatcgagctgggcaagaagttcaacatcaagacccggctgagctacttcagcatgaaccacgaccccgtgaagatccccaacctgctggacgacaccatctacaacgacaccgagggcttcaacatcgagagcaaggacctgaagagcgagtacaagggccagaacatgcgggtgaacaccaacgccttccggaacgtggacggcagcggcctggtgagcaagctgatcggcctgtgcaagaagatcatcccccccaccaacatccgggagaacctgtacaaccggaccgcctg

SEQ ID NO: 22 - protein, 594 aa, Homo sapiens
MASSTPSSSATSSNAGADPNTTNLRPTTYDTWCGVAHGCTRKLGLKICGFLQRTNSLEEKSRLVSAFRERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 23 - DNA, 1785 bp, Homo sapiens
  source 1..1785: mol_type = genomic DNA
atggcgtcttcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcagggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatatctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaa

SEQ ID NO: 24 - protein, 254 aa, unidentified
  REGION 1..254: note = Saporin S6 protein
MVTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPK

SEQ ID NO: 25 - DNA, 765 bp, unidentified
  misc_feature 1..765: note = Saporin S6 gene
  source 1..765: mol_type = genomic DNA
atggtaacatcaattaccctcgaccttgtcaaccctaccgctggacaatactcatccttcgtagataaaataaggaacaatgtcaaagaccctaatctcaagtacggtggtacagatatcgctgtcatcggccctccctcaaaagaaaaattcctcaggataaactttcaatcttccaggggaacggtctcacttggacttaagagggataatctgtatgtggtggcttatttggcaatggataatactaacgtgaatcgcgcatattactttcggagtgaaataacaagtgcagagagcaccgcattgttccccgaagcgacaactgcgaaccagaaagccttggaatacacagaggactatcagtccatcgaaaagaacgcgcagataactcaaggagaccagagtaggaaagaactcggcctcggcatcgatctcttgagtaccagcatggaggccgtgaacaaaaaggctagggtagttaaagatgaagccaggttcctcctgatagctatacagatgaccgctgaggccgccaggtttaggtatatccaaaaccttgtgatcaagaattttcccaacaaattcaacagcgagaataaggtgatacagtttgaggtaaactggaaaaaaatcagcaccgctatttatggggacgcgaaaaacggagtattcaataaagactacgatttcggcttcgggaaagttcgccaagttaaagacttgcaaatgggactgttgatgtatctcggcaaaccgaagtga

SEQ ID NO: 26 - protein, 217 aa, unidentified
  REGION 1..217: note = Nitroreductase nfnB protein
MDIISVALKRHSTKAFDASKKLTPEQAEQIKTLLQYSPSSTNSQPWHFIVASTEEGKARVAKSAAGNYVFNERKMLDASHVVVFCAKTAMDDVWLKLVVDQEDADGRFATPEAKAANDKGRKFFADMHRKDLHDDAEWMAKQVYLNVGNFLLGVAALGLDAVPIEGFDAAILDAEFGLKEKGYTSLVVVPVGHHSVEDFNATLPKSRLPQNITLTEV

SEQ ID NO: 27 - DNA, 654 bp, unidentified
  misc_feature 1..654: note = Nitroreductase nfnB gene
  source 1..654: mol_type = genomic DNA
atggacataatttccgtcgctctcaagcgacattcaacaaaggcgtttgacgcttcaaaaaagttgacacctgaacaggcggaacagatcaagacgttgctccagtattccccgtcttctactaacagccagccctggcactttatcgtggcttccacagaggagggcaaagctcgagtagctaaaagcgcggcaggcaactatgtattcaatgagcgaaagatgcttgatgcgtcccatgtcgtggtattttgtgcgaagacagctatggacgatgtgtggcttaagctggtggtagatcaagaggatgccgacggcaggttcgccaccccagaagccaaggctgctaatgacaagggtcgcaaattttttgcggatatgcacaggaaagatctccacgacgatgccgaatggatggcaaaacaggtctacctcaacgtaggtaactttttgcttggtgtggctgctttgggtctggatgcggtgccgatcgagggctttgatgcggctatacttgatgctgagttcggcttgaaggaaaaaggatatacttcccttgtcgtagtgcccgttgggcatcatagtgtcgaggactttaacgctaccctgccgaaatctaggctcccgcaaaacataacactcacggaggtctga

SEQ ID NO: 28 - protein, 465 aa, synthetic construct
  REGION 1..465: note = C. botulinum neurotoxin of type A linked to the signal peptide ofsyntaxin 1a
MHHHHHHQFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSIMIIICCVILGIVIASTVGGIF

SEQ ID NO: 29 - DNA, 1401 bp, synthetic construct
  misc_feature 1..1401: note = C. botulinum neurotoxin of type A linked to the signal peptide ofsyntaxin 1a
  source 1..1401: mol_type = other DNA
atgcaccaccaccaccaccaccagttcgtgaataaacaattcaattataaagacccagtgaatggtgttgacatagcatacatcaaaatcccgaacgtgggacagatgcaaccggtgaaagccttcaagattcataacaagatctgggttattcctgaacgggacacttttaccaaccctgaagaaggtgacctgaaccctcctccagaggctaagcaggttcctgtttcctactacgattcaacttatctgagcactgataacgaaaaggataattaccttaagggagttaccaaactgttcgagcgcatttatagcacagacctcggcagaatgctgctgaccagcatagtgcggggaattccattttgggggggcagcacaatcgacacggagttgaaggtcatcgatacgaattgcatcaacgtgatacaaccagatggctcttacagatccgaggaactgaacctggtgatcatcggcccctctgctgatataatccaattcgaatgcaaaagcttcggtcacgaggtgctgaatcttacccggaacggatacgggtccacccagtacatacgctttagtcccgactttacattcggcttcgaggaaagtcttgaagtggacacgaatccactgctgggagctggcaagttcgccactgatcctgccgttacacttgctcatgaactgattcatgctggacaccggctttatgggatagctataaatccgaatagagtctttaaggttaacacaaatgcctactacgaaatgtctggccttgaggtttcattcgaggagcttaggacctttggaggccacgacgctaaattcatcgactctctgcaggagaatgagtttcggctgtactactacaacaagtttaaggacattgccagtactctgaacaaggctaagagcatcgtcgggaccacagccagcctccaatatatgaaaaacgtgttcaaggaaaagtaccttctgtcagaagacacatcaggaaaattctcagtcgacaaactgaaatttgacaagctgtacaagatgctgactgaaatatacacagaggacaacttcgtgaagttttttaaagtcctgaacagaaagacttacttgaacttcgacaaagccgtctttaaaatcaatatcgtcccaaaggttaattacactatctatgacggattcaatctcagaaacacgaacttggctgccaacttcaatggacagaacacagagatcaacaacatgaattttactaagcttaaaaatttcacaggcctgttcgagttctataagcttctttgcgtccgaggcatcattacatccatcatgatcataatctgctgtgtgatattgggcatagtgattgcatccaccgtggggggcatttttgcctag

SEQ ID NO: 30 - protein, 469 aa, synthetic construct
  REGION 1..469: note = C. botulinum neurotoxin of type B linked to the signal peptide ofsyntaxin 1a
MHHHHHHPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVIMIIICCVILGIVIASTVGGIFA

SEQ ID NO: 31 - DNA, 1410 bp, synthetic construct
  misc_feature 1..1410: note = C. botulinum neurotoxin type B linked to the signal peptide ofsyntaxin 1a
  source 1..1410: mol_type = other DNA
atgcaccaccaccatcatcatcccgtgaccatcaacaatttcaactataatgaccctatcgataacaacaacatcattatgatggagccccctttcgcccgcggcaccgggagatactacaaggccttcaaaataaccgataggatctggatcatcccagagaggtacaccttcgggtacaagcctgaggactttaataaatcaagcggtatctttaatagggatgtgtgtgaatactacgaccctgactatctcaataccaacgacaaaaagaatatcttccttcagactatgatcaagcttttcaatcgaattaagagtaagccgcttggtgagaaactgctggagatgatcataaacggcatcccctacctcggagatcgccgcgttccgctggaagagtttaacactaatatcgcaagcgtcactgtaaataaactcatcagcaacccgggggaagtggaaaggaagaagggaatctttgctaacctgattatctttggaccaggcccagtgttgaatgaaaacgagaccatcgacatcgggatccagaaccactttgcatcacgagaggggtttggggggattatgcagatgaagttctgccccgagtacgtgtcagtgttcaataacgtgcaggaaaacaaaggagcatccatcttcaatcgccgaggctacttctctgatcctgctctcatcctcatgcacgagctcattcacgtgctgcacggactttatggcatcaaggtggacgacctgcctattgtgccgaatgaaaagaagttcttcatgcagagtactgatgccatccaggctgaggaactgtacactttcgggggccaggacccatccattatcaccccaagtactgataagtcaatctatgacaaagttctgcagaacttccgcggaatcgtggataggctcaacaaagtgctggtgtgtattagcgaccccaacattaacatcaatatttacaagaacaaattcaaggacaaatataaattcgtggaggactctgagggcaagtattcaattgacgtggagagcttcgacaaactgtacaaaagcctgatgttcggtttcacagagaccaacatagcagagaactataagattaaaactcgcgcgagctacttttcagattcactgcctccggtgaaaatcaagaacctcctggataatgagatctataccatagaagaaggatttaacatttccgacaaggacatggaaaaggagtaccggggacagaacaaggccatcaacaaacaggcctatgaagaaatcagcaaggagcacctcgccgtctacaaaattcaaatgtgcaaaagcgtcataatgattattatctgctgcgtaatcctggggatagtgatcgcttccaccgtaggcggcatcttcgcctga

SEQ ID NO: 32 - protein, 476 aa, synthetic construct
  REGION 1..476: note = C. botulinum neurotoxin of serotype C linked to the signalpeptide of VAMP2
MHHHHHHPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNMMIILGVICAIILIIIIVYFST

SEQ ID NO: 33 - DNA, 1431 bp, synthetic construct
  misc_feature 1..1431: note = C. botulinum neurotoxin of serotype C linked to the signalpeptide of VAMP2
  source 1..1431: mol_type = other DNA
atgcaccaccatcatcatcatcctattacaatcaacaattttaattattctgacccagtggacaacaaaaacatactttaccttgatacccacctcaatactctggcgaacgagccagagaaagcgttccgcataacaggaaacatatgggtgattcctgacagattcagtcgaaatagtaacccaaacctgaacaagcctccgagggttacatcccctaagtccggttattacgaccccaattacctgtctacagatagcgataaagatcctttcctgaaagagatcattaaactgttcaaacgaataaactcccgcgaaatcggggaggaactcatttatcgattgtccacggacatccctttccctggtaataacaacaccccgattaatacctttgacttcgacgtcgactttaactctgtggatgtgaagactcggcagggtaacaactgggttaaaactgggtcaatcaacccgtctgtcataattacaggccctagggagaatataattgatccggagaccagcacctttaaattgactaataatactttcgccgcacaggaggggttcggcgccctgtctatcatatcaatcagtccccgatttatgctgacctactctaatgcaactaacgacgtcggggaaggtcggtttagcaaaagtgagttctgcatggacccgatcctcatactgatgcacgagttgaaccatgcaatgcataatctgtatggaatcgctattcccaacgatcagacaatatcttccgtcacgtcaaacattttctattctcagtataatgtgaaactggaatatgctgagatctacgcctttggtggccccacaattgacctgattccaaagagcgccaggaagtacttcgaggaaaaggcacttgattattataggagcatcgcaaagcgcctgaacagcatcacaacggccaacccaagctctttcaacaaatatataggcgaatacaagcaaaaactcattagaaaatacaggtttgtggtggaaagcagcggagaggtaaccgtaaaccgcaacaaattcgtggagctgtacaacgaactgactcagatctttacggaattcaattacgctaagatctacaatgtgcagaaccggaagatttacctgtccaatgtttacacacctgtcactgctaatattctcgatgacaatgtgtacgacattcagaatggcttcaacatccccaagtctaacctgaacgtgctgttcatgggccagaaccttagccgcaatcctgcgctgcgcaaagtcaaccctgagaatatgctgtatctcttcacgaagttctgtcacaaggccatagacggtagaagtctttataatatgatgattatactgggggtgatctgcgcgattatccttattattatcattgtatacttctctacatga

(19) United States
(12) Patent Application Publication
(10) Pub. No.: US 2026/0258426 A1
(43) Pub. Date: Sep. 3, 2026

(54) Viral Vectors For Treating Neurogenic Detrusor Overactivity

(71) Applicants: Assistance Publique - Hopitaux De Paris, Paris (FR); Universite De Versailles-St Quentin En Yvelines, Versailles Cedex (FR)

(72) Inventors: François GIULIANO, Paris (FR); Alberto L. EPSTEIN, Lyon (FR); Olivier LE COZ, Le Mesnil saint denis (FR); Alejandro ARANDA, Pamplon (ES)

(21) Appl. No.: 19/381,855

(22) Filed: Nov. 6, 2025

Related U.S. Application Data
(62) Division of application No. 17/866,022, filed on Jul. 15, 2022, now Pat. No. 12,516,326.
Application No. 17/866,022 is a continuation of application No. 16/312,867, filed on Jun. 23, 2017, now Pat. No. 11,414,666.

(30) Foreign Application Priority Data
Jun. 23, 2016 (EP) 163057656

(51) Int. Cl. C12N 15/113; A61K 38/16; A61K 38/17; A61K 38/45; A61K 38/48; A61K 38/51; A61P 13/06; C12N 15/86

(52) U.S. Cl. CPC C12N 15/1138; A61K 38/164; A61K 38/1793; A61K 38/45; A61K 38/4893; A61K 38/51; A61P 13/06; C12N 15/86; C12Y 304/24069; C12Y 401/01015; C12N 2310/11; C12N 2320/31; C12N 2320/32; C12N 2710/16643; C12N 2830/008; C12N 2830/40

(57) ABSTRACT

The present invention provides a method and a pharmaceutical composition for the treatment of the NDO comprising the viral expression vector carrying a transcription cassette that harbors transgene(s) inhibiting/silencing neurotransmission or synaptic transmission of afferent neurons.

### CROSS-REFERENCE TO RELATED APPLICATIONS

This application is a Divisional of U.S. application Ser. No. 17/866,022, filed on Jul. 15, 2022, which is a Continuation of U.S. application Ser. No. 16/312,867, filed on Dec. 21, 2018 (now U.S. Pat. No. 11,414,666), which is the National Phase under 35 U.S.C. § 371 of International Application No. PCT/EP2017/065587, filed on Jun. 23, 2017, which claims the benefit under 35 U.S.C. § 119(a) to patent application Ser. No. 16/305,765.6, filed in Europe on Jun. 23, 2016, all of which are hereby expressly incorporated by reference into the present application.

### REFERENCE TO ELECTRONIC SEQUENCE LISTING

The Sequence Listing submitted via EFS, in compliance with 37 CFR § 1.52(e)(5), is incorporated herein by reference. The sequence listing XML file submitted via EFS contains the file “2022-07-15_SequenceListing_3493-0679PUS2.xml”, created on Jul. 14, 2022, which is 70,118 bytes in size.

The present invention is directed to a viral expression vector and a pharmaceutical composition thereof that selectively modulates or silences the afferent nerves of the bladder, as a gene therapy strategy for the treatment of neurogenic detrusor overactivity (NDO).

In particular, the present invention is related to the field of control of urine storage and bladder emptying or micturition, which is dependent upon the activity of two functional units in the lower urinary tract: (1) a reservoir (the urinary bladder) and (2) an outlet consisting of the bladder neck, urethra, and striated muscles of the external urethral sphincter (EUS) (Fowler et al. 2008; Morrison et al. 2005). These structures are controlled by three sets of efferent peripheral nerves: sacral parasympathetic (pelvic nerves), thoracolumbar sympathetic (hypogastric nerves and lumbo-sacral sympathetic chain), and somatic nerves (pudendal nerves) distributed bilaterally (de Groat 1986; Morrison et al. 2005). These nerves consist of efferent axons originating at thoracolumbar and sacral spinal levels. Parasympathetic efferent nerves contract the bladder and relax the urethra. Sympathetic efferent nerves relax the bladder and contract the urethra. Somatic efferent nerves contract the EUS. These nerves also contain afferent neurons that transmit information from the lower urinary tract to the lumbosacral spinal cord. The cellular bodies of the afferent neurons of the human lower urinary tract are located in the S2-S4 and T11-L2 dorsal root ganglia (DRG). Sensations of bladder fullness are conveyed to the spinal cord by the pelvic and hypogastric nerves, whereas sensory input from the bladder neck and the urethra is carried in the pudendal and hypogastric nerves.

A similar segmental organization occurs in nonhuman primates, cats and dogs. In rats, cellular bodies of the afferent neurons of pelvic, pudendal and hypogastric nerves are located in the L6-S1 and T11-L2 DRG respectively. The neural pathways that control lower urinary tract function are organized as simple on-off switching circuits that maintain a reciprocal relationship between the urinary bladder and the urethral outlet. Storage reflexes are activated during bladder filling and are organized primarily in the spinal cord, whereas voiding is mediated by reflex mechanisms that are organized in the brain (Fowler et al. 2008). Throughout bladder filling, the parasympathetic innervation of the detrusor is inhibited and the smooth and striated parts of the urethral sphincter are activated, preventing involuntary bladder emptying. This process is organized by urethral reflexes known collectively as the ‘guarding reflex’. They are activated by bladder afferent activity that is conveyed through the pelvic nerves, and are organized by interneuronal circuitry in the spinal cord (Fowler et al. 2008).

NDO refers to a condition in which abnormal bladder function is observed in patients with neurological diseases, such as cerebrovascular disease or cerebral infarction, brain or spinal cord injury due to trauma, multiple sclerosis, Parkinson's disease, congenital malformation e.g. spina bifida, or disease e.g. hereditary spastic paraplegia of the central nervous system, peripheral neuropathy, and various spinal lesions, that is, spinal cord compression and injury due to vertebra(e) fracture, cervical and lumbar spondylosis, spondylosis deformans, spondylolisthesis, spinal stenosis, vertebral disk hernia and the like.

NDO is characterized by involuntary detrusor (bladder) contractions during the filling phase, which may be spontaneous or provoked due to a relevant neurological condition. It is often associated to bladder-sphincter dyssynergia.

NDO due to spinal cord injury (SCI) is the most severe form of NDO. Immediately after SCI there is a period of spinal shock lasting for 2-12 weeks during which the bladder is areflexic, accountable for complete urinary retention. Then, a spinal micturition reflex progressively develops that is responsible for NDO. For SCI patients, these impairments lead to urinary incontinence and increase in bladder pressure, which, if untreated, can damage upper urinary tract and precipitate renal failure. Urinary incontinence is associated with a significant burden and severely impairs quality of life. In SCI patients, recurrent urinary tract infections due to incomplete bladder emptying and renal failure remain the first cause of rehospitalization and second cause of mortality respectively. SCI disrupts voluntary control of voiding as well as the normal reflex pathways that coordinate bladder and sphincter functions. In suprasacral spinal lesion, NDO results of the unmasking of a segmental reflex at the level of the sacral cord, mediated by bladder afferent nociceptive C-fibers (de Groat and Yoshimura, 2006). These silent C-fibers become mechano-sensitive and initiate automatic micturition reflex after SCI. This reflex is facilitated after elimination of supraspinal control. Plasticity occurs in bladder afferents and is associated with changes in the properties of ion channels and electrical excitability of afferent neurons, and appears to be mediated in part by neurotrophic factors released in the spinal cord and the peripheral target organs. Overall, the neurobiological substrate for NDO comprises functional alterations in bladder urothelium and sub-urothelium as well as increased afferent sensory messages to the spinal cord, originating in the bladder. Exacerbated afferent bladder stimuli, resulting from hypertrophy and hyperactivity of non-myelinated type-C bladder afferent neurons, are the main mechanisms causing NDO in SCI subjects.

Standard of care for the treatment of NDO consists in inhibiting efferent neurotransmission at the detrusor level. Accordingly, NDO patients are currently treated with antimuscarinics, which block the activity of the muscarinic acetylcholine receptors thereby inhibiting detrusor contractions, and/or repeated intradetrusor injection of Clostridium botulinum neurotoxin A (BoNT-A), again to block detrusor contractions by acting on bladder efferents. Both treatments must be combined with intermittent bladder catheterization (5-6 times/day).

BoNT-A injections suppress the formation of SNARE complex, blocking the fusion of neurotransmitter-filled vesicles with the plasma membrane of efferent neurons and their release during exocytosis. Accordingly, injection of BoNT-A is used as medication for treating patients with overactive bladder from neurogenic origin or not. For example, PCT patent applications WO 99/03483 and WO 2010/022979 disclose the use of BoNT-A injection to prevent a nerve from stimulating its target tissue, e.g. a muscle, a gland, or another nerve, for the treatment of various urinary disorders.

WO2013/180799 discloses the use of a viral vector encoding a modified botulinum neurotoxin, thereby producing a protein that has improved binding properties to its human receptors. Following production in cell lines, once recovered and purified from the supernatants, this neurotoxin can be locally applied to treat a condition associated with unwanted neuronal activity such as NDO. However, these vectors are not conceived for a gene therapy approach.

Nevertheless, injection of botulinum neurotoxins presents the inconvenient of toxin diffusion, which is largely due to diffusion of toxins to other regions of the body. The adverse effects range from transient non-serious events such as ptosis and diplopia to life-threatening events even death. In addition, for NDO these injections must be repeated in average every 6 months because of decreased efficacy overtime.

Because NDO, with or without bladder-sphincter dyssynergia, caused by supra sacral spinal lesions is due to the emergence of an abnormal reflex mediated by bladder afferences (a□ and c fibers), an alternative approach for the treatment of NDO has been developed by Brindley (Brindley et al 1986). This approach combines posterior sacral rhizotomies and sacral anterior roots stimulation (SARS). This treatment appeared to be one of the most effective therapeutic methods for NDO caused by complete suprasacral spinal lesions: sacral rhizotomies permanently increases the compliance of the bladder and eliminates hyperactivity of the detrusor—and detrusor-sphincteric dyssynergia—which are the main causes of renal failure and urinary incontinence, while implantation of a stimulator of the anterior spinal roots enables the patient to elicit and to control micturition.

Deafferentation by posterior sacral rhizotomies, as proposed by Brindley (1986), consists of the complete surgical transection of all afferent neural fibers to the spinal S2-S4 segments, including those providing sensory input from the detrusor muscle. In this way, the sensory stimuli from the detrusor muscle cannot reach anymore the central nervous system, and consequently, reflex activities generated by the central nervous system causing uncontrolled bladder contractions can be inhibited. The procedure is necessary to prevent exacerbated reflex activities of detrusor and allows larger amount of urine to be stored at low bladder pressure. However, bladder deafferentation obtained from extensive, non-selective, irreversible pelvi-perineal deafferentation by posterior sacral rhizotomies (S2-S4) has many pitfalls and drawbacks, as it is responsible for loss of remaining pelvi-perineal sensation if present, impairing orgasm if present, reflex erection and ejaculation if present, and reflex micturition and defecation if present, and possibly facilitating bedsore because of loss of skin sensory innervation. In addition, the magnitude of neurosurgical procedure makes it expensive and can be responsible for cerebrospinal fluid fistulas and in the long-term for Charcot spinal arthropathy.

Consequently, there is a need for a new strategy to treat NDO in case of supraspinal lesion, targeting specifically its pathophysiology i.e. the abnormal spinal reflex mediated by bladder afferences, but without affecting other afferent neurons conveyed in the same nerves, while sparing the bladder efferent neurons. The strategy we propose is a gene therapy approach resulting in selective molecular bladder deafferentation, to restore continence and micturition in NDO patients when combined with sacral anterior roots stimulation. This has been achieved by a new strategy requiring a viral expression vector able to deliver therapeutic transgene(s) presenting:
capacity to inhibit/silence neurotransmission or synaptic transmission of afferent neurons;
high selectivity, notably for the afferent neurons of the bladder;
high efficiency;
stability of expression over time; and
absence of off-target denervation.

In the context of the present invention, the inventors surprisingly found that, following injection of the viral expression vector in the bladder wall, it is possible to obtain selective and stable transgenes expression in the afferent neurons of the bladder, using a viral expression vector that stably expresses over time proteins and/or transcripts to treat NDO, by specifically inhibiting/silencing neurotransmission or synaptic transmission of bladder afferent neurons at the spinal cord level.

The present invention provides a method and a pharmaceutical composition for the treatment of the NDO comprising the viral expression vector carrying a transcription cassette that harbors transgene(s) inhibiting/silencing neurotransmission or synaptic transmission of afferent neurons. Preferably, the method and a pharmaceutical composition according to the invention comprise a viral expression vector carrying a transcription cassette that harbors transgene(s) disrupting SNARE complex, and/or ribosomal complex, and/or activating GABA(A) receptors, and/or inducing conditionally targeted neuron ablation, when transcribed, that inhibit/silence neurotransmission or synaptic transmission of bladder afferent neurons.

The term “transcription cassette” as used herein refers to any nucleic acid sequence containing a promoter and a downstream coding sequence or transgene, which expression is driven by said promoter, which is followed by a polyadenylation signal. The term “transgene” refers to a particular nucleic acid sequence encoding for a RNA and/or a polypeptide or a portion of a polypeptide to be expressed in a cell into which the nucleic acid sequence is introduced. The term “transgene” includes (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced. By “mutant form” is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell, or the transgene may include both a leader peptide or signal sequence plus a membrane anchor peptide or even be a fusion protein between two naturally occurring proteins or part of them, such that the transgene will remain anchored to cell membranes.

As used herein, the term “ribosomal complex” refers to a complex which is essentially composed of the subunits of ribosomes, such as 80S and 70S subunits that catalyzes the synthesis of proteins, referred as translation.

In a first aspect, the present invention thus provides a viral expression vector comprising at least:
a) one promoter selectively active in afferent neurons of the bladder,
b) one transcription cassette comprising a nucleotide sequence operably linked to said promoter, wherein said nucleotide sequence silences or inhibits the transduction of the neurotransmitter signal in postsynaptic cell when transcribed, and
c) one sequence conferring long-term expression, such as that known as LTE (Lokensgard et al, 1997) and/or containing DNA insulators (Amelio et al, 2006) from the HSV-1 genome, operably linked to said transcription cassette.

In preferred embodiment, the nucleotide sequence of viral expression vector according to the invention silences or inhibits neurotransmission or synaptic transmission when transcribed or translated by disrupting the SNARE complex, and/or the ribosomes complex, and/or by activating GABA(A) receptors, and/or by inducing conditionally targeted neuron ablation.

In a preferred embodiment, the nucleotide sequence of viral expression vector according to the invention, when transcribed, disrupts at least one of the proteins selected from VAMP, SNAP-25 or syntaxin 1a, which are part of the SNARE complex, or codes for the protein GAD67 or for an active fragment thereof, or codes for a protein disrupting the ribosomes complex or for an active fragment thereof, or codes for a protein inducing conditionally targeted neuron ablation, or for an active fragment thereof.

In a particular embodiment, the said protein disrupting the ribosomes complex according to the invention is a wild-type or a modified ribosome inactivating protein (RIP) or an active fragment thereof, preferentially said RIP are selected from RIP of type 1 or type 2, preferentially RIP of type 1 are selected from saporin, gelonin, dianthin, trichosanthin; and RIP of type 2 are selected from ricin, volkensin and abrin, more preferentially said RIP of type 1 is saporin S6 or an active fragment thereof.

The term “protein inducing conditionally targeted neuron ablation” relates to a protein which converts innocuous prodrug substrates, such as metronidazole (MTZ), into cytotoxic DNA crosslinking agents—providing cell-specific ablation of the targeted cell type i.e. afferent neuron of the bladder. Example of such protein inducing conditionally targeted neuron ablation are nitroreductases (NTR).

The protein inducing conditionally targeted neuron ablation according to the invention is therefore selected from the group consisting of a wild-type or a modified NTR or an active fragment thereof. Preferentially, said NTR is selected from the group consisting of a wild-type or a modified oxygen-insensitive NAD(P)H nitroreductases or an active fragment thereof, more preferentially said NTR is selected from the group consisting of a wild-type or a modified E. coli nitroreductases, even more preferentially said NTR is a wild-type or a modified E. coli nfnB or an active fragment thereof.

The term “viral vector” or “viral expression vector” as used herein refers to a nucleic acid vector that includes at least one element of a virus genome and may be packaged into a viral particle. In the context of the present invention, the term “viral vector” has to be understood broadly as including nucleic acid vector (e.g. DNA viral vector) as well as viral particles generated thereof. According to the present invention the viral expression vector is an adeno-associated virus (AAV) vector or a herpes simplex virus (HSV) vector, preferably a HSV-1 vector or a HSV-2 vector, even more preferably a defective viral vector derived from HSV-1. As used herein the term “defective viral vector” shall refer to viral vectors that are missing genes or parts of genes necessary to complete successfully the viral life cycle.

According to the present invention, the term “AAV” refers to the Adeno-Associated Virus itself or to derivatives thereof including recombinant AAV vector particles. Furthermore, as used herein, the term “AAV” includes many different serotypes, which have been isolated from both human and non-human primate samples. Preferred AAV serotypes are the human serotypes, more preferably human AAV of serotypes 2, 5 and 9, most preferably human AAV of serotype 5, which is the serotype displaying the highest level of neurotropism.

According to the present invention, the term “defective viral vector derived from HSV” refers both to defective recombinant HSV vectors and amplicon HSV vectors. The terms “defective recombinant HSV”, as used herein, describes a helper-independent vector, the genome of which comprises at least complete deletions of the genes coding for two essential proteins, known as ICP4 and ICP27. The ICP4 gene is present in two copies, located in the inverted repeated sequences known as c and c′ of the virus genome, and both copies of this gene are deleted. The gene encoding ICP27 is located in the unique long (UL) sequence of the virus genome. Preferentially, helper-independent vectors according to the invention carry the therapeutic transcription cassette(s) embedded into the LAT (Latency Associated Transcripts) locus (Berthomme et al. 2000 and Berthomme et al. 2001), which is a repeated locus that is contained in the inverted repeated sequences known as b and b′ of the virus genome. More preferentially, the transcription cassette is placed either between the Latency Associated Promoter (LAP) and the Long-Term Expression (LTE) region (site 1), or between the LTE region and the DNA insulator (INS) sequence present downstream of the LTE (site 2) (as shown in FIG. 1). Defective recombinant HSV-1 vectors according to the present invention carry transcription cassette(s) expressing the different transgenes above described in order to inhibit/silence neurotransmission, i.e. expressing wild type or modified light chain botulinum toxins, and/or antisense RNA (AS-RNA) targeting SNARE proteins, and/or GAD67, and/or RIPs, and or NTRs, all of them driven by long-term DRG-specific promoters as described in the present invention. The b and b′ sequences of the virus genome are also known as TRL (Terminal Repeat L) and IRL (Internal Repeat L) respectively, while the c′ and c sequences are also known as IRS (Internal Repeat S) and TRS (Terminal Repeat S), where L and S refer respectively to the unique long (L) and unique short (S) sequences of the HSV-1 genome. Moreover, helper-independent vectors according to the invention can comprise additional deletions in genes encoding non-essential proteins such as ICP34.5, UL55, UL56, and UL41 proteins. These defective HSV vectors are multiplied in cell lines expressing simultaneously the proteins ICP4 and ICP27 (Marconi et al, 2010).

WO 2006/050211 discloses the use of a defective HSV-1 vector for gene therapy of pain.

However, the vectors according to the invention differ from the vector described in WO 2006/050211 in several significant respects, which are important in regard to the usefulness and efficacy of the vectors according to the invention. Most important, transgenic transcription cassettes according to the invention are introduced into the LAT locus, as this region contains both the LTE and the DNA insulator sequences (INS) that confer long-term expression to the DRG-specific promoters driving transgene expression in transcription cassettes according to the invention, whereas the vector described in WO 2006/050211 was conceived and proved for short-term action and, therefore, their transcription cassettes are driven by ubiquitous promoters and were not introduced into the LAT regions.

By “Amplicon or amplicon vector” it is meant a helper-dependent vector, the genome of which lacks most or all HSV genes coding for virus proteins. The genome of amplicon vectors is a concatemeric DNA composed of multiple copies in tandem of a plasmid—known as the amplicon plasmid—that carries one origin of DNA replication and one packaging signal from HSV-1 genome, in addition to transgenic DNA (i.e. transcription cassettes) of interest. Amplicon plasmids according to the present invention carry transcription cassettes expressing the different transgenes above described in order to inhibit/silence neurotransmission, i.e., expressing wild type or modified light chain botulinum toxins, and/or interfering RNA (RNAi) targeting SNARE proteins, and/or GAD67, and/or RIPs, and/or NTRs, all of them driven by long-term promoters, preferentially a long-term DRG-specific promoters as described in the present invention (see FIG. 2).

In a preferred embodiment, the vector according to the invention is a defective recombinant vector lacking at least the genes coding for the essential proteins ICP4 and ICP27, preferentially a vector lacking both ICP4 and ICP27. This vector can lack other genes, coding for non-essential proteins, such as ICP34.5, UL55, UL56 and/or UL41 gene proteins, and carries the DRG-specific transcription cassette(s), described in FIG. 7, embedded into the LAT regions of the vector genome.

In another embodiment, the vector according to the invention is an amplicon vector carrying the above described transcription cassettes driven by long-term DRG-specific promoters, as described in other parts of this document.

In a preferred embodiment, the transcription cassette according to the invention is introduced into the LAT locus.

The expression “Recombinant DNA” as used herein describes a nucleic acid molecule, i.e., a polynucleotide of genomic, cDNA, viral, semisynthetic, and/or synthetic origin, which, by virtue of its origin or manipulation, is not associated with all or a portion of the polynucleotide with which it is associated in nature. The term “recombinant” as used with respect to virus means a virus carrying a recombinant genome or a genome that has been manipulated to introduce mutations, deletions or one or more heterologous polynucleotides, including genes. The term “recombinant” as used with respect to a protein or polypeptide, means a polypeptide produced by expression of a recombinant nuclei acid. The term “recombinant” as used with respect to a host cell means a recombinant vector that carries recombinant DNA within the host cell or a cell that contains recombinant DNA inserted in its genome. The term “infection” refers to the ability of a viral vector to enter into a host cell or subject.

Defective vectors derived from HSV allow to infect neighbouring sensory neurons and establish latent infections in the nucleus of these neurons, located in the trigeminal or the dorsal root ganglia (DRG), depending on the site of infection. In particular, HSV-1 naturally infects sensory neurons and establishes lifelong latent infections in the nucleus of these neurons. It could thus be hypothesized that following injection in the bladder wall, the vectors, such as vector derived from HSV-1, will reach the sensory DRG innervating the bladder from where they will stably express the therapeutic transgene, provided that adequate bladder afferent neuron-specific promoters drive their expression. However, HSV-1 can also infect and establish latent infections in autonomic neurons (Furuta et al., 1993; Warren et al. 1978), and preliminary results demonstrate that this is actually the case when the vector is inoculated into the bladder. Therefore, it is mandatory that expression from the vectors be utterly controlled by afferent-specific promoters, also called selective promoters or selective afferent neuron-specific promoters, in order to obtain significant transgene expression only in these neurons (i.e. afferent neuron), thus avoiding expression in autonomic, also called efferent, neurons. Selective molecular or biochemical (as opposite to surgical) deafferentation of bladder afferent neurons is the most critical aspect of the present invention as it is important to preserve remaining pelvi-perineal sensation if present, orgasm if present, reflex erection and ejaculation if present, and reflex micturition and defecation if present, all of which are conveyed by sensory nerves of the pelvis that do not originate in the bladder. Further, selective bladder deafferentation would also allow to preserve bladder efferent neurons, which could be later stimulated by electrical stimulation for example via electrodes. Some studies describe the use of HSV-1-based vectors in which the transcription cassettes comprise either transient (Miyazato et al., 2009) or long-term (Puskovic et al., 2004; Miyagawa et al., 2015; WO 2015/009952A1) promoters. However, the promoters used in the studies of Puscovic (LAP2), Miyazato (HCMV promoter) and Miyagawa (artificial CAG promoter) are non-selective, leading to expression of their transgenes in many cell types, including autonomic neurons, brain neurons, and non-neuronal cells. In contrast, by combining viral regulatory sequences and afferent neuron-specific cellular promoters, some of the vectors according to the present invention enable a significantly higher afferent neuron-specific expression of the transgenes of interest (see FIG. 13).

The vectors used in the practice of the invention include at least one promoter selectively active in afferent neurons that is operationally linked to nucleotides (usually DNA) encoding an RNA molecule. By “operationally linked” it is meant herein that, in the vector, the promoter is associated with the nucleotides encoding the RNA in a manner that allows the promoter to drive transcription (i.e. expression) of the RNA from the nucleotides. Transcription of RNA from, e.g. a DNA template is well-understood.

A “promoter,” as used herein, is a DNA regulatory region capable of binding RNA polymerase in a mammalian cell and initiating transcription of an operably linked downstream (3′ direction) sequence. For purposes of the present invention, a promoter sequence includes at least the minimum number of bases or elements necessary to initiate transcription of a gene of interest at levels detectable above background. Within the promoter sequence is a transcription initiation site, as well as RNA polymerase binding domains. Eukaryotic promoters will often, but not always, contain “TATA” boxes and other DNA motifs, such as “CAT” or “SP1” boxes. The promoter according to the invention comprises DNA sequence starting at least 2 kb, preferably 3 kb, more preferably 4 kb upstream to the initiation site of the messenger codifying for specific, relevant gene products. These sequences preferably contain known promoters' sequences elements, such as specific transcription binding sites, and distal sequences upstream of the gene, containing additional regulatory elements.

By “active selectively in afferent neurons” it is meant herein that the promoter is active mainly or only in the afferent neurons, preferably in afferent neurons of the bladder and drives transcription (i.e. expression) of the RNA.

Also, those of skill in the art will recognize that many such mammalian afferent neuron specific promoters are known, and additional afferent neuron specific promoters are continually being discovered. All such afferent neuron specific promoters are encompassed by the present invention. However, many cell-specific promoter candidates have been shown to display selectivity only when they express from their endogenous location in the cellular chromosomes (McCart et al., 2002; Vassaux et al., 1996). There is no way to predict how these promoters will behave when introduced into the genome of a non-integrative expression vector, such as HSV vectors. Notably, it cannot be anticipated whether afferent neuron-specific promoters will retain the same afferent neuron-specific activity. This is both because (a) the nucleosomes bound to the promoter could differ in several respects (for example they can be in a repressive or a permissive configuration) according to the location of the promoter (in the chromosomes versus in the extra-chromosomal vector genome, or even between different positions in cellular chromosomes) and also (b) because the accessibility of positive or negative transcription factors could also differ. This means that every promoter candidate should be thoroughly studied in each specific setting (i.e. episomal vector vs. chromosomal location) to establish whether it retains or not its afferent neuron-specific activity when placed into the vector genome, as we experimentally did (see results in Example 11 and FIG. 13).

In a preferred embodiment, the promoter according to the invention is selected from promoters of genes coding for sensory neuroreceptors, such as Transient Receptor Potential Vanilloid 1 (TRPV1) or Transient Receptor Potential cation channel subfamily M member 8 (TRPM8), or from promoters of genes coding for sensory neuromodulators or sensory neurotransmitters, such as the promoters of Substance P, PACAP, Calcitonin Gene Related Peptide (CGRP) of SEQ ID NO: 3 or SEQ ID NO: 4. Preferentially, promoter of genes coding for sensory neuroreceptors according to the invention is a promoter of the TRP gene family, more preferentially the promoter TRPV1 of SEQ ID NO: 1 or TRPM8 of SEQ ID NO: 2. Preferentially, promoters of genes coding for sensory neuromodulators or sensory neurotransmitters according to the invention is the CGRP of SEQ ID NO: 3 or SEQ ID NO: 4, or the promoter of genes involved in neurite outgrowth and stress response in sensory neurons, preferably the promoter of the gene encoding advillin (ADVL) of SEQ ID NO: 5 or SEQ ID NO: 6.

The viral expression vector of the invention is directed more particularly to vertebrate, preferably to mammals, more preferably primates and humans. Therefore, those skilled in the art will recognize that such promoters are specific to species and would be able to select homologous sequences of a particular species of interest. In particular, the promoters according to the invention are human homolog of rat TRPV1 of SEQ ID NO: 1 or human TRPM8 of SEQ ID NO: 2, or rat CGRP of SEQ ID NO: 3, or human CGRP of SEQ ID NO: 4, or rat advillin of SEQ ID NO: 5 or human advillin of SEQ ID NO: 6, amongst others.

By “long-term expression sequence” or “long-term expression element (LTE)” it is meant a nucleotide sequence operably linked to the transcription cassette included in the sequence of the viral expression vector, allowing to sustain the expression of a gene product for more than 15 to 45 days or 30 to 45 days, preferably 45 to 90 days, more preferably 90 to 365 days, even more preferably 365 days to several years or even more preferably during the life of the patient.

Long-term expression (LTE) sequences were identified in HSV-1 as a region of the latency-associated transcripts (LAT), which originate from the LAT-associated promoter (LAP). This LTE is located downstream of the LAT transcription start site. Indeed, viruses harboring a DNA fragment 3′ of the LAT promoter maintained detectable promoter expression throughout latency (Lokensgard et al, 1997, Berthomme et al., 2000, 2001). Preferably, the LTE is comprised between about 1.5 kb to about 3 kb downstream of the LAT transcription start site (Perng et al., 1996). More recently, additional sequences, known as DNA insulators, have also been described both upstream and downstream the LTE region (Amelio et al., 2006). These sequences also contribute to provide long-term expression to a given transcription cassette probably by inhibiting epigenetic silencing, and also will be incorporated in the present invention as part of the LTE elements, to confer long-term expression to the transcription cassette. Interestingly, sequences conferring long-term expression to the transcription cassette (both the LTE and the DNA insulator sequences) can be placed either upstream and/or downstream the transcription cassette.

Those of skill in the art will recognize that other LTE-like sequences, as well as other DNA insulator sequences, have been described and are continually being discovered. All such LTE-like sequences and DNA insulator sequences are encompassed by the present invention.

In preferred embodiment, the viral expression vector of the invention comprises at least one nucleotide sequence that is transcribed into a non-coding nucleotides sequence inhibiting the synthesis of at least one protein selected from VAMP, SNAP-25 and syntaxin, which are part of the SNARE complex.

The SNARE complex (soluble N-ethylmaleimide-sensitive factor attachment protein receptor) is one of the two key components of the membrane fusion machinery with the SM (Sec1/Munc18) proteins. The SNARE complex comprises the vesicle-associated “v-SNAREs” (Vesicle Associated Membrane Proteins, VAMPs, particularly VAMP1, 2 and 3) and the target membrane-associated “t-SNAREs” Syntaxins (Syn-1, 2, 3, and 4) and Synaptosome-Associated Protein of 25 kDa (SNAP-25) that assemble into complexes to mediate different fusion events.

Therefore, one embodiment of the present invention is directed to methods able to silence a specific gene and/or to disrupt the corresponding encoded protein (a “gene of interest” or “targeted gene” or “selected gene”). By “silencing” a gene, we mean that expression of the gene product is reduced or eliminated, in comparison to a corresponding control gene that is not being silenced. Those of skill in the art are familiar with the concept of comparing results obtained with control vs. experimental results. Without being bound by theory, it is believed that silencing is characterized by specific mRNA degradation or mRNA block in translation after the expression of a non-coding complementary sequence such as antisense RNA (asRNA), a small hairpin RNA (shRNA), a microRNA (miRNA), or any other form of interfering RNA (iRNA) into cells.

As herein used, the term “antisense” relates to unmodified or chemically modified single-stranded nucleic acid molecules which are relatively short in general and which are able to hybridize to a unique sequence in the total pool of targets present in cells, the sequence of said nucleic acid molecule being complementary by virtue of Watson-Crick bp hybridization, to a specific mRNA and is able to inhibit said mRNA expression and then induce a blockade in the transfer of genetic information from DNA to protein.

In the context of the invention, “RNA interference” (hereinafter referred to as RNAi) is interpreted as a process by which a double stranded RNA (dsRNA) with a given sense nucleic sequence leads to the breakdown of all messenger RNA (mRNA) comprising said nucleic sequence, in a manner specific to said nucleic sequence. Although the RNAi process was originally demonstrated in Caenorhabditis elegans, it is now clear that the RNAi process is a very general phenomenon, and inhibition of human genes by RNAi has been achieved.

The process of RNAi can be achieved using small interfering RNA (or siRNA). These siRNAs are dsRNA of less than 30 nucleotides long, comprising in their sense sequence a sequence that is highly complementary to a fragment of the target mRNA. When a siRNA crosses the plasma membrane, the reaction of the cell is to destroy the siRNA and all the sequences comprising a highly complementary sequence. Thus, an mRNA with a fragment that is highly complementary to the siRNA sequence will be destroyed, the expression of this gene being thus inhibited.

shRNA may be also used as inhibitor according to the present invention. As used herein, an “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a conventional shRNA forms a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

In general, “reduced or eliminated” refers to a reduction or elimination of detectable amounts of the gene product by an amount in the range of at least about 10% to about 100%, or preferably of at least about 25% to 100%, or more preferably about 50% to about 100%, and most preferably from about 75% to about 100%. If desired, a reduction or elimination may be determined by any of several methods that are well known to those of skill in the art, and may vary from case to case, depending on the gene that is being silenced. For example, such a reduction or elimination of the expression of the gene may be determined by quantification of the gene product (e.g. by determining the quantity of a protein, polypeptide or peptide that is made) or quantification of an activity of the gene product (e.g. an activity such as signaling or transport activity, activity as a structural component of the cell, activity such as enzymatic activity, etc.), or by observation and quantification of a phenotypic characteristic of the targeted cell in comparison to a control cell (e.g the presence or absence of a protein using specific antibodies). Any suitable means to determine whether or not a targeted gene has been silenced may be used.

In one embodiment, the non-coding nucleotides sequence according to the invention is selected from antisense RNA (asRNA), a small hairpin RNA (shRNA), a micro RNA (miRNA), or any other interfering RNA (iRNA), which inhibits the synthesis of at least one protein selected from VAMP, SNAP-25 and syntaxin.

In one embodiment, the viral expression vector comprises at least one nucleotide sequence that is transcribed into an asRNA inhibiting the synthesis of VAMP, SNAP-25 and/or syntaxin. In particular, the sequences of the asRNA used in the context of the present invention are VAMP2 antisense of SEQ ID NO: 7, SNAP25 antisense of SEQ ID NO: 8 and syntaxin antisense of SEQ ID NO: 9.

In a particular embodiment, the viral expression vector comprises at least one nucleotide sequence that is transcribed into an shRNA inhibiting the synthesis of VAMP, SNAP-25 and/or syntaxin.

In another embodiment, the viral expression vector comprises at least one nucleotide sequence that is transcribed into an miRNA inhibiting the synthesis of VAMP, SNAP-25 and/or syntaxin.

The RNA molecule that is encoded by the construct of the present invention ultimately forms a double-strand RNA molecule within the cell in which it is transcribed. In general, one strand of the double-strand RNA structure will be in the range of from about 10 to about 30 ribonucleotides in length, and preferably from about 19 to about 25 ribonucleotides in length. In the case of asRNA, one of the double-strand RNA structure will be in the range of from about 100 to several hundreds of ribonucleotides in length. It could actually be as long as the target mRNA. Those of skill in the art will recognize that several viable strategies exist for forming such double-strand RNA.

Moreover, provision of multiple viral vectors with the same afferent neuron-specific promoter but which encode different silencing RNAs may be used within the practice of the invention.

Further, it should be possible to express more than one silencing RNA in a single viral vector, driven by a single afferent neuron-specific promoter, or by more than one promoter arranged in tandem (e.g. two or more promoters). Thus, the invention contemplates using a single viral vector for silencing more than one gene.

In another embodiment, the viral expression vector according to the invention comprises at least one nucleotide sequence coding for a wild-type or a modified toxin disrupting the SNARE complex or the ribosome complex or for an active fragment thereof.

Advantageously, the active fragment of the toxin is a bacterial neurotoxin, preferentially said bacterial neurotoxin is the light chain of said bacterial neurotoxin. In particular, the sequences of the toxins light chains used in the context of the present invention are the protein sequence light chain of the botulinum neurotoxin A (BoNT-A) of SEQ ID NO: 10 (coding nucleotides sequence SEQ ID: 11), the protein sequence light chain of the botulinum neurotoxin B (BoNT-B) of SEQ ID NO: 12 (coding nucleotides sequence SEQ ID: 13), the protein sequence light chain of the botulinum neurotoxin C1 (BoNT-C1) of SEQ ID NO: 14 (coding nucleotides sequence SEQ ID: 15), the protein sequence light chain of the botulinum neurotoxin E3 (BoNT-E3) of SEQ ID NO: 16 (coding nucleotides sequence SEQ ID: 17), the protein sequence light chain of the botulinum neurotoxin F1 (BoNT-F1) of SEQ ID NO: 18 (coding nucleotides sequence SEQ ID: 19) and the protein sequence light chain of the tetanic neurotoxin (TeNT) of SEQ ID NO: 20 (coding nucleotides sequence SEQ ID: 21).

In preferred embodiment, the viral expression vector according to the invention comprises at least one nucleotide sequence coding for a wild-type or a modified GAD67 protein or for an active fragment thereof, preferentially nucleotide sequence coding for a wild-type GAD67 protein of SEQ ID NO: 22 (coding nucleotides sequence SEQ ID: 23) or an active fragment thereof.

In preferred embodiment, the viral expression vector according to the invention comprises at least one nucleotide sequence coding for a wild-type or a modified RIP or for an active fragment thereof, preferentially said RIP is Saporin S6 protein of SEQ ID NO: 24 (coding nucleotides sequence SEQ ID: 25) or an active fragment thereof.

In preferred embodiment, the viral expression vector according to the invention comprises at least one nucleotide sequence coding for a wild-type or a modified NTR or an active fragment thereof, preferentially said NTR is nitroreductase nfnB protein of SEQ ID NO: 26 (coding nucleotides sequence SEQ ID: 27) or an active fragment thereof.

As used herein, the term “coding sequence” refers to a ribonucleic acid (e.g., RNA) sequence that, when it is translated, produces the polypeptide of interest. The polypeptide can be encoded by a full-length coding sequence or by any portion of the coding sequence so long as the desired activity or functional properties (e.g., enzymatic activity, ligand binding, signal transduction, etc.) of the full-length or fragment is retained.

In one embodiment, the invention relates to a viral expression vector that comprises at least one nucleotide sequence coding for a wild-type or a modified botulinum neurotoxin of Clostridium botulinum of any serotype or for an active fragment thereof, preferably the light chain of Clostridium botulinum neurotoxin of any serotype.

In another embodiment, the invention is directed to a viral expression vector that comprises at least one nucleotide sequence coding for a wild-type or a modified tetanus neurotoxin of Clostridium tetani or for an active fragment thereof, preferably the light chain of Clostridium tetani neurotoxin.

Clostridial neurotoxins are produced by various species of the genus Clostridium, for example several strains of C. botulinum and C. tetani. When Clostridium toxin molecules enter into the neuron, the light chain disrupts the proteins that form the SNARE complex located at the presynaptic nerve terminal. This prevents the neurotransmitter filled synaptic vesicles from attaching to the presynaptic membrane, therefore inhibiting exocytosis of the neurotransmitter from the presynaptic nerve terminal. At present, there are eight different classes of the neurotoxins known: tetanus toxin and botulinum neurotoxin in its serotypes A, B, C, D, E, F and G, all of which share homology and similar molecular structures. Within said serotypes, sub-types are also well documented, such as subtypes A₁-A₃, B₁-B₃, etc.

Botulinum neurotoxin serotypes A, C, and E cleaves the SNAP-25 protein located on the plasma membrane of the presynaptic nerve terminals. Because SNAP-25 is necessary for the fusion of neurotransmitter-filled vesicles with the plasma membrane and their release during exocytosis, its cleavage causes a highly specific blockade of vesicular neurotransmitter release at somatic and autonomic presynaptic nerve terminals. Botulinum neurotoxin serotypes B, D, F, and G cleave the synaptobrevin (VAMP) protein, so that the vesicles cannot fuse to the cell membranes. Each botulinum neurotoxin or its light chain fragment cleaves one of the SNARE proteins except for botulinum neurotoxin C, or its light chain fragment, which cleaves both SNAP25 and syntaxin 1a. Preferably, according to the invention the serotypes of botulinum neurotoxin are A, B, C, E and F.

The structure of Clostridial neurotoxins has been well-documented (Habermann et al, 1986; Sugiyama et al 1980); each of these documents is hereby incorporated in its entirety by reference thereto]. In this regard, Clostridial neurotoxins comprise two polypeptide chains, the heavy chain (H-chain), which has a molecular mass of approximately 100 kDa, and the light chain (L-chain), which has a molecular mass of approximately 50 kDa, joined together by a disulphide bond.

The different serotypes of botulinum toxin vary in the animal species that they affect and in the severity and duration of the paralysis they evoke. For example, it has been determined that botulinum toxin type A is 500 times more potent, as measured by the LD₅₀ in mice, than botulinum toxin type B. Additionally, botulinum toxin type B has been determined to be non-toxic in primates at a dose of 480 U/kg which is about 12 times the primate LD₅₀ for botulinum toxin type A.

Naturally, botulinum toxin binds with high affinity to neurons, is translocated into the neuron and blocks the release of neurotransmitters.

In a particular embodiment, the invention is directed to a viral expression vector that comprises at least one nucleotide sequence coding for a wild-type or a modified tetanus neurotoxin of Clostridium tetani or for an active fragment thereof to cleave the protein VAMP-2.

In a particular embodiment, the invention is directed to a viral expression vector that comprises at least one nucleotide sequence coding for a wild-type or a modified botulinum neurotoxin of Clostridium botulinum of serotype B, D, F, and G or for an active fragment thereof to cleave the protein VAMP-2.

In a particular embodiment, the invention is directed to a viral expression vector that comprises at least one nucleotide sequence coding for a wild-type or a modified botulinum neurotoxin of Clostridium botulinum of serotype A and E or for an active fragment thereof to cleave the protein SNAP-25.

In a preferred embodiment, the invention is directed to a viral expression vector that comprises at least one nucleotide sequence coding for a wild-type or a modified botulinum neurotoxin of Clostridium botulinum of serotype C or for an active fragment thereof to cleave the proteins SNAP25 and syntaxin 1a.

In a preferred embodiment, the nucleotide sequence of transgene according to the invention codes for a wild-type or a modified protein silencing or inhibiting the transduction of the neurotransmitter signal in postsynaptic cell which is fused to a signal peptide domain. The signal peptide according to the invention is selected according to the intracellular compartment where transcript or protein targeted to silence or inhibit the transduction of the neurotransmitter signal in postsynaptic cell is located. Therefore, those skilled in the art will recognize that such signal peptides are specific to intracellular compartment and would be able to select the appropriate corresponding nucleotide sequences to be fused to the nucleotide sequence coding for the protein silencing or inhibiting neurotransmission or synaptic transmission according to the invention. In particular, the signal peptides according to the invention comprise at least the luminal, transmembrane or cytoplasmic domains of proteins selected from VAMP2 or Syntaxin 1a.

In a particular embodiment, fusion protein according to the invention comprises a signal peptide domain selected from luminal, transmembrane or cytoplasmic signal peptide domains, preferentially the luminal, transmembrane or cytoplasmic signal peptide domains of the SNARE proteins, the substance P or CGRP sequences. Such signal peptide domains include notably the signal peptide of syntaxin 1a (BoNTB-STX) of SEQ ID NO: 30 (coding nucleotides sequence SEQ ID: 31) and the signal peptide of VAMP2 (BoNTC-VAMP) of SEQ ID NO: 32 (coding nucleotides sequence SEQ ID: 33). Thus, according to a particular embodiment of the invention, the fusion protein comprises a modified bacterial neurotoxin, such as e.g., a modified botulinum neurotoxin, and a signal peptide such as e.g., the signal peptide of syntaxin 1a (BoNTA-STX) of SEQ ID NO: 28 or (BoNTB-STX) of SEQ ID NO: 30 (coding nucleotides sequence SEQ ID: 31) and the signal peptide of VAMP2 (BoNTC-VAMP) of SEQ ID NO: 32 (coding nucleotides sequence SEQ ID: 33).

In one specific embodiment, the fusion protein according to the invention comprises the wild-type or modified Clostridium botulinum neurotoxin of serotype A, B, C, E or F linked to the signal peptide of syntaxin 1a, preferentially the fusion protein comprises the wild-type or modified Clostridium botulinum neurotoxin of serotype A linked to the signal peptide of syntaxin 1a (BoNTA-STX) of SEQ ID NO: 28 (coding nucleotides sequence SEQ ID: 29) or the fusion protein comprises the wild-type or modified Clostridium botulinum neurotoxin of serotype B linked to the signal peptide of syntaxin 1a (BoNTB-STX) of SEQ ID NO: 30 (coding nucleotides sequence SEQ ID: 31).

In one specific embodiment, the fusion protein according to the invention comprises the wild-type or modified Clostridium botulinum neurotoxin of serotype A, C and E linked to the signal peptide of VAMP2, preferentially the fusion protein comprises the wild-type or modified Clostridium botulinum neurotoxin of serotype C linked to the signal peptide of VAMP2 (BoNTC-VAMP) of SEQ ID NO: 32 (coding nucleotides sequence SEQ ID: 33).

In one specific embodiment, the fusion protein according to the invention comprises the wild-type or modified Clostridium botulinum neurotoxin of any serotype linked to the signal peptide of Substance P.

In one specific embodiment, the fusion protein according to the invention comprises the wild-type or modified Clostridium botulinum neurotoxin of any serotype linked to the signal peptide of CGRP sequence.

The present invention is also directed to a viral expression vector according to the invention, comprising at least:
a) one nucleotide sequence coding for a wild type or modified neurotoxin of Clostridium tetani or botulinum or for an active fragment thereof; and/or
b) one nucleotide sequence whose transcripts inhibit the synthesis of the protein VAMP, SNAP-25 and/or syntaxin; and/or
c) one nucleotide sequence coding for a wild type or modified GAD67 protein or for an active fragment thereof; and/or
d) one nucleotide sequence coding for a wild type or modified RIP or for an active fragment thereof; and/or
e) one nucleotide sequence coding for a wild type or modified NTR or for an active fragment thereof.

In a preferred embodiment, the viral expression vector according to the invention comprises:
i. one said long-term expression sequence operably linked to two transcription cassettes according to the invention; or
ii. two long-term expression sequences both operably linked to one said transcription cassette according to the invention; and wherein:
   a) one transcription cassette according to the invention harbors a coding sequence according to the invention, and the second transcription cassette according to the invention harbors a sequence that is transcribed into a non-coding nucleotide according to the invention; or
   b) both transcription cassettes according to the invention harbor a nucleotide sequence coding for a non-coding nucleotides sequence according to the invention; or
   c) both transcription cassettes according to the invention harbor a nucleotide sequence coding for a wild type or modified neurotoxin of Clostridium tetani and/or botulinum or for an active fragment thereof, or for a wild type or modified GAD67 protein or for an active fragment thereof; or for a wild type or modified RIP or for an active fragment thereof, or for a wild type or modified NTR or for an active fragment thereof.

In a particular embodiment, the invention relates to a viral expression vector, wherein
i. one said long-term expression (LTE) sequence is operably linked to two transgenic transcription cassettes according to the invention; or
ii. two separated long-term expression (LTE) sequences are each operably linked to one said transcription cassette according to the invention;
   and wherein:
a) one transcription cassette according to the invention harbors a sequence coding for a bacterial neurotoxin, a GAD67, a RIP, or a NTR according to the invention, and the second transgenic transcription cassette according to the invention harbors a sequence that is transcribed into a non-coding nucleotides sequence according to the invention that inhibit the synthesis of the protein VAMP, SNAP-25 and/or syntaxin; or
b) both transcription cassettes according to the invention harbor a nucleotide sequence coding for a non-coding nucleotides sequence according to the invention that inhibit the synthesis of at least one protein selected from VAMP, SNAP-25 and/or syntaxin; or
c) both transgenic transcription cassettes according to the invention harbor a promoter and a nucleotide sequence coding for a wild type or modified neurotoxin of Clostridium tetani and or botulinum or for an active fragment thereof, or a wild type or modified GAD67 protein or for an active fragment thereof, or a wild type or modified RIP or for an active fragment thereof, or a wild type or modified NTR or for an active fragment thereof.

In a preferred embodiment, the invention relates to a viral expression vector, wherein at least one of said transgenic transcription cassettes according to the invention harbors a promoter and a sequence coding for the wild-type protein GAD67 or for an active fragment thereof.

In a more preferred embodiment, the invention relates to a viral expression vector, wherein one of the said transgenic transcription cassettes according to the invention harbors a promoter and a sequence coding for the wild-type protein GAD67 or for an active fragment thereof, and one of the said transgenic transcription cassettes according to the invention harbors a promoter and a sequence coding for a wild type or modified neurotoxin of Clostridium tetani and or botulinum or for an active fragment thereof.

In a preferred embodiment, the invention relates to a viral expression vector, wherein at least one of said transgenic transcription cassettes according to the invention harbors a promoter and a sequence coding for the wild-type RIP or for an active fragment thereof.

In a more preferred embodiment, the invention relates to a viral expression vector, wherein one of the said transgenic transcription cassettes according to the invention harbors a promoter and a sequence coding for the wild-type RIP or for an active fragment thereof, and one of the said transgenic transcription cassettes according to the invention harbors a promoter and a sequence coding for a wild type or modified neurotoxin of Clostridium tetani and or botulinum or for an active fragment thereof, and/or a sequence coding for the wild-type protein GAD67 or for an active fragment thereof, and/or a sequence coding for the wild-type NTR of for an active fragment thereof.

In a preferred embodiment, the invention relates to a viral expression vector, wherein at least one of the transgenic transcription cassettes according to the invention comprises a promoter and a sequence coding for the wild-type NTR or for an active fragment thereof.

In a more preferred embodiment, the invention relates to a viral expression vector, wherein said viral expression vector comprises at least 2 transgenic transcription cassettes, wherein:
at least one of the said transgenic transcription cassettes according to the invention comprises a promoter and a sequence coding for the wild-type NTR or for an active fragment thereof; and
At least one of the said transgenic transcription cassettes according to the invention comprises a promoter and a sequence coding for a wild type or modified neurotoxin of Clostridium tetani and or botulinum or for an active fragment thereof, and/or a sequence coding for the wild-type protein GAD67 or for an active fragment thereof, and/or a sequence coding for the wild type RIP or for an active fragment thereof.

In a second aspect, the invention relates to a composition comprising the viral expression vector of the present invention for use as a medicament.

In a third aspect, the invention is directed to a pharmaceutical composition comprising at least one viral expression vector according to the invention.

Advantageously, the pharmaceutical composition according to the invention is used for the treatment of the NDO.

The invention also relates to a pharmaceutical composition comprising:
a) at least one viral expression vector comprising at least one nucleotide sequence transcribed into a non-coding nucleotides sequence, preferably selected from antisense RNA (asRNA), a small hairpin RNA (shRNA) or a microRNA (miRNA), more preferably antisense RNA (asRNA), to inhibit the synthesis of at least one protein selected from VAMP, SNAP-25 and syntaxin; and/or
b) at least one viral expression vector comprising at least one nucleotide sequence coding for a wild-type or a modified bacterial neurotoxin disrupting the SNARE complex or for an active fragment thereof, preferably the light chain of a bacterial neurotoxin, and wherein the said bacterial neurotoxin is advantageously the neurotoxin of Clostridium tetani and/or Clostridium botulinum of any serotype, preferably serotypes A, B, C, E and F; and/or
c) at least one viral expression vector comprising at least:
   one nucleotide sequence coding for a wild type or modified neurotoxin of Clostridium tetani or botulinum or for an active fragment thereof, and
   one nucleotide sequence whose transcripts inhibit the synthesis of the protein VAMP, SNAP-25 and/or syntaxin; and/or
d) at least one viral expression vector according to the invention, wherein
   i. one said long-term expression (LTE) sequence is operably linked to two transgenic transcription cassettes according to the invention; or
   ii. two long-term expression (LTE) sequences are each operably linked to one said transgenic transcription cassette according to the invention; and wherein:
      one transgenic transcription cassette according to the invention harbors a promoter and sequence coding for said neurotoxin, and the second transgenic transcription cassette according to the invention harbors a promoter and a sequence nucleotide inhibiting the synthesis of the protein VAMP, SNAP-25 and/or syntaxin; or
      both transgenic transcription cassettes according to the invention harbor a promoter and a nucleotide sequence coding for a non-coding nucleotides sequence inhibiting the synthesis of at least one protein selected from VAMP, SNAP-25 and/or syntaxin; or
      both transgenic transcription cassettes according to the invention harbor a nucleotide sequence coding for a wild type or modified neurotoxin of Clostridium tetani and/or botulinum or for an active fragment thereof for simultaneous, separated or staggered use for treating NDO.

In a particular embodiment, the pharmaceutical composition according to the invention, further comprises at least one viral expression vector comprising at least one nucleotide sequence coding for the wild-type protein GAD67 and/or RIP and/or NTR, or for an active fragment thereof.

In a particular embodiment, the pharmaceutical composition according to the invention, comprises at least one viral expression vector comprising at least one nucleotide sequence coding for the wild-type protein GAD67 or for an active fragment thereof, and/or at least one nucleotide sequence coding for a wild type or modified neurotoxin of Clostridium tetani and or botulinum or for an active fragment thereof, and/or for the wild-type RIP or for an active fragment thereof, and/or for the wild-type NTR or for an active fragment thereof.

In a fourth aspect, the present invention relates to a kit comprising at least one viral expression vector or the pharmaceutical composition according to the invention, or the pharmaceutical composition according to the invention, and an electrical stimulation system comprising electrodes to be implanted on the sacral anterior roots, such as S2-S3-S4, to apply intermittent stimulation pulse trains in order to achieve a sustained detrusor muscle contraction with intervals of urethral sphincter relaxation allowing urine to flow.

By “electrical stimulation” it is meant herein that an electrical stimulation is applied, via electrodes, in bursts of a few seconds, separated by longer gaps, to sustain pressure in the bladder, while allowing the external urethral sphincter to relax rapidly between bursts, causing urine to flow during these gaps. The preferred electrical stimulation system is the Finetech-Brindley stimulator (ref 6 à 19 in Ren et al, 2015).

The invention further relates to a method for the treatment of patient suffering from NDO comprising the steps of:
a) preparing at least one viral expression vector according to the invention;
b) injecting the viral expression vector of step a) in the bladder wall (detrusor muscle);
c) implanting electrical stimulation system via electrodes implanted on the sacral anterior roots, such as S2-S4 or S3-S4, to elicit by stimulation in bursts of a few seconds, separated by longer gaps, a sustained pressure in the bladder, while allowing the external urethral sphincter to relax rapidly between bursts, causing urine to flow.

The following examples merely intend to illustrate the present invention.

### DESCRIPTION OF THE FIGURES

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A-1C. Genome of recombinant defective HSV-1 vectors.

FIG. 1A: The upper part of the figure describes the backbone of the HSV-1 genome used in this invention. The HSV-1 genome contains two unique regions, known as Unique Long (UL) and Unique Short (US), each bordered by repeated inverted sequences, known as Terminal Repeat L/Inverted Repeat L (TRL/IRL) and Inverted Repeat S/Terminal Repeat S (IRS/TRS). TRL/IRL are also denominated ab/b′a′, whereas IRS/TRS are also denominated a′c′/ca. The genome therefore starts and ends by the direct repeat sequence ‘a’. The black square in UL indicates that the gene coding for the essential ICP27 protein is deleted in the vector used in this invention. Similarly, the two black squares in the IRS/TRS repeats, indicate that the two genes coding for the essential ICP4 protein are also deleted. The white circle in UL, as well as the two white circles in the IRS and TRS regions, indicate the origins of DNA replication of HSV-1 (respectively OriL and two copies of OriS). Other genes, coding for non-essential proteins, such as UL41, UL55 and UL56, can be also deleted. In addition, both copies of the IE4/5 promoters localized in the IRS and TRS are modified in such a way (deletion of one TAATGARAT sequence) that these promoters express with early kinetics (instead of immediate-early kinetics as in the wild-type virus genome).

FIG. 1B: The middle part of the figure shows a detail of the b′a′a′c′ region of the virus genome, indicating in particular the localization of LAT locus in the IRL region, which contains the gene expressing the latency associated transcripts (LAT).

FIG. 1C: The bottom part of the figure shows the detailed structure of the 5′ part of the LAT locus that carries the therapeutic DRG-specific transcription cassettes (indicated in the figure as the arrow labeled Transgene). This locus includes an upstream DNA insulator (INS) sequence, the Latency Associated Promoter (LAP), a region conferring Long-Term Expression (LTE) and a downstream DNA insulator (INS). The therapeutic DRG-specific transcription cassette is introduced either between the LAP and the LTE (site 1) or between the LTE and the second DNA insulator (site 2). Other genes in the vicinity of LAT are also indicated in B (arrows). The different DRG-specific transcription cassettes that are introduced in the LAT region to generate the recombinant vectors are shown in FIG. 3A-3B. It should be stressed that the region b′a′a′c′ is identical to the inverted caab region, which forms when the virus genome becomes circularized in the cell nucleus at the beginning of infection. This means that both copies of ICP4 are deleted and that the transgenic transcription cassette can be introduced in both copies of the LAT locus.

FIG. 2. Genome of amplicon vectors (the amplicon plasmid).

Amplicon plasmids are standard E. coli plasmids generally carrying three modules: (1) The bacterial module, which contains the Col E1 sequence for plasmid replication in bacteria, and a gene conferring resistance to an antibiotic, generally ampicillin (in black). (2) The amplicon module, which contains an HSV-1 origin of DNA replication, generally OriS (O), and a packaging signal (a) allowing amplification and packaging of a concatemeric form of the amplicon plasmid; in addition, this module generally express a reporter protein (in our case, either a GFP protein, or a fusion GFP/Renilla luciferase (rLuc) protein) driven by the HSV-1 immediate early promoter IE4/5. (3) The third module is the transcription unit, containing the DRG-specific transcription cassette (grey arrow labeled Transgene) placed between the LTE and INS sequenced, designed to inhibit or silence neurotransmission stably and selectively in sensory neurons, as described in this invention. The different transcription cassettes that are introduced into the amplicon plasmid to generate the amplicon vectors are shown in FIG. 3A-3B.

FIG. 3A-3B. FIG. 3A. This figure represents the region of the genome of amplicon vectors used in this invention that carries the two eukaryotic transcription cassettes. One of them expresses the reporter GFP (or the fusion GFP-rLuc) gene under the control of the IE4/5 immediate-early promoter of HSV-1. The second transcription cassette expresses any of the therapeutic functions that inhibit or silence neurotransmission, as described in this invention. A DRG-specific promoter drives expression of the transcription cassettes, whereas the whole cassette is surrounded by sequences conferring long-term expression (black squares). FIG. 3B. This figure shows some of the transcription cassettes used in this study to demonstrate the efficacy and selectivity of the genetic constructs. These are: vector A: HCMV-TeNT light chain, (LC); vector B: HCMV-BoNT-A (LC); vector C: HCMV-BoNT-C (LC); vector D: SNAP25 antisense RNA; vector E: HCMV-Luciferase; vector F: TRPV1-Luciferase. BoNT-A (LC) and BoNT-C (LC) are fusion proteins that express a C-terminal HIS-tag, as no efficient anti-BoNT antibodies are available. HCMV is a strong and ubiquitous viral promoter, whereas TRPV1 is a DRG-selective promoter. Other vectors, expressing other botulinum toxins, or fusion SNARE/light chain toxins, or antisense RNA addressed to other SNARE proteins, or the human GAD67 protein, or a RIP protein such as Saporin S6, are not shown in this Figure.

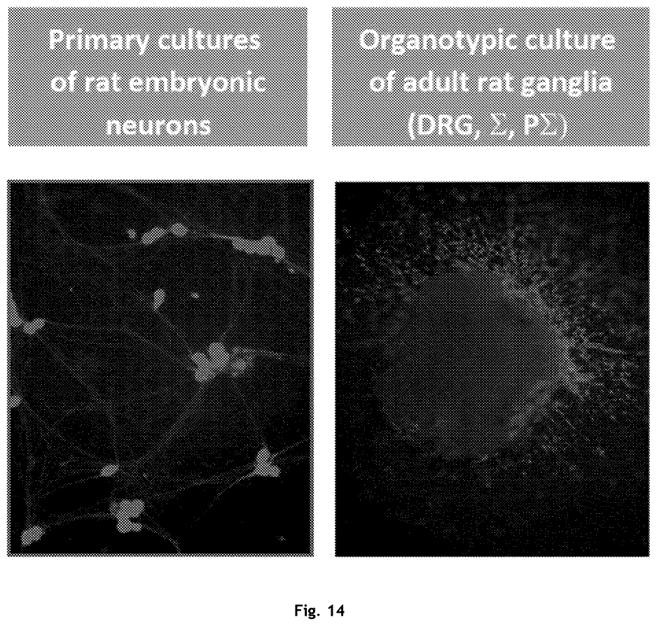

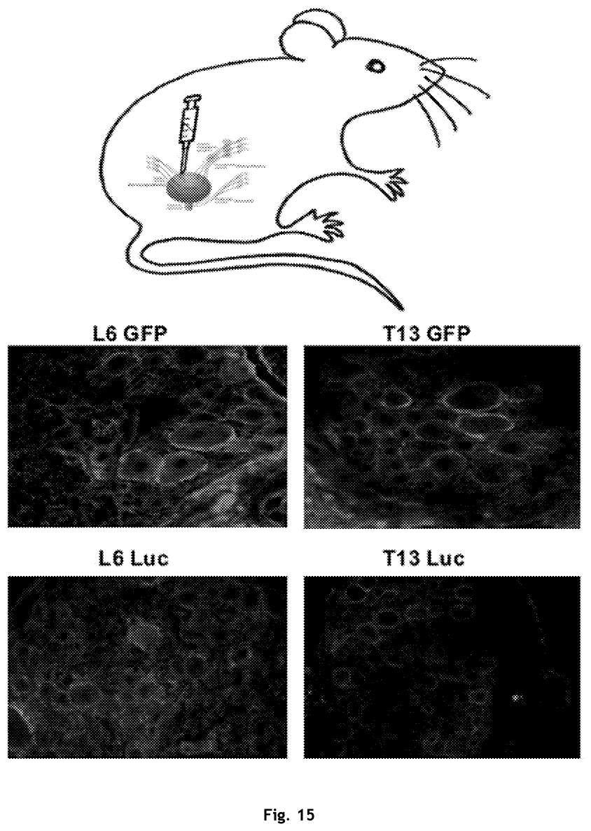

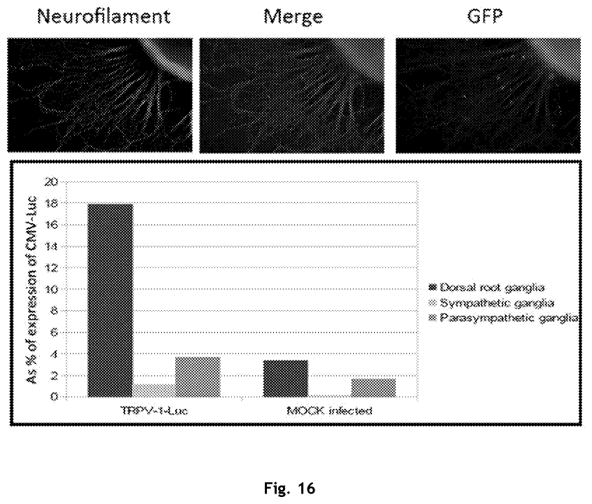

FIG. 4 shows the expression of BoNT-A (LC), BoNT-C (LC) and TeNT (LC) in Gli36 (a cell line derived from a human glioblastoma) and BHK21 (hamster fibroblast cells) cell lines. Gli36 and BHK21 cells were infected with the amplicon vectors HCMV-Luc, HCMV-BoNT-A (LC), HCMV-BoNT-C (LC), or HCMV-TeNT (LC) (shown in FIG. 2B). Infected cells were then fixed and expression of the toxins was demonstrated using specific antibody in a Western assay. Anti-TeNT antibodies were used to reveal TeNT (LC); anti-HIS antibodies were used to reveal both BoNT-A (LC) and BoNT-C (LC).

FIG. 5 shows that the toxin TeNT (LC) expressed in Gli36 cells and present in cell extracts possesses proteolytic activity with respect to VAMP2. Gli36 cells were infected with the amplicon vector expressing HCMV-TeNT (LC) at a multiplicity of infection (MOI) of 1. The infection was terminated 2 days later and protein extracts were prepared. These extracts were incubated in a suitable buffer (50 mM Hepes, 400 mM NaCl, 5 mM dithiothreitol and 2 μM ZnSO4) containing the target protein of TeNT, i.e., VAMP2. After incubation for 24 h at 37° C. with 2.5, 5, and 10 μL of cell extracts, westerns blots were performed using anti-VAMP2 antibody to reveal the proteolytic activity of TeNT (LC) expression.

FIG. 6A-6B. FIG. 6A shows that at 48 hours post-infection (hpi) of human neuroblastoma SH-S5Y5 cells with amplicon vectors expressing HCMV-BoNT-A (LC) (see FIG. 3B), there is a significant decrease in cellulo of SNAP25 protein levels relative to the control cells infected with a vector expressing luciferase (HCMV-Luc) or not infected (Mock). Protein levels were detected by Western blot assay using anti-SNAP25 antibodies. Note that BoNT-A (LC) cleaves SNAP25 into two fragments. The antibodies used in these experiments recognize both the native SNAP25 protein (upper band) and the large fragment of the cleaved protein (lower band). FIG. 6B shows that at 48 hours post-infection (hpi) of human neuroblastoma SH-S5Y5 cells with amplicon vectors expressing HCMV-BoNT-C (LC) (see FIG. 3B), there is a significant decrease in cellulo of both SNAP25 and Syntaxin (STX) protein levels relative to the control cells infected with a vector expressing luciferase (HCMV-Luc) or not infected (Mock). Protein levels were detected by Western blot assay using anti-SNAP25 and anti-STX antibodies. Note that BoNT-C (LC) cleaves SNAP25 into two fragments. The antibodies used in these experiments recognize both the native SNAP25 protein (upper band) and the large fragment of the cleaved protein (lower band).

FIG. 7A-7C. Transcription cassettes carried by recombinant and amplicon vector genomes.

FIG. 7A-7C shows some of the transcription cassettes that are carried and expressed by the recombinant and amplicon HSV-1 vectors. These transcription cassettes are classified into three families. Members of the A2 family are transcription cassettes expressing different therapeutic gene products (proteins or antisense RNA or miRNA), driven by the strong and ubiquitous HCMV promoter. Vectors carrying the A2 transcription cassettes are used to study the impact of these gene products on neurotransmission (cleavage of SNARE proteins and inhibition of neurotransmitter release), thus allowing to select the most efficient transgenes in the context of this invention. Members of the A5 family are transcription cassettes expressing the reporter gene firefly luciferase (fLuc) driven by different DRG-selective candidate promoters. These vectors are used to study the intensity, selectivity, and duration of expression in cultured neurons and in explanted peripheral ganglia, thus allowing identifying the most selective vectors in the context of this invention. Finally, members of the A8 family of vectors express therapeutic transcription cassettes (therapeutic gene products driven by DRG-selective promoters), thus allowing selecting vectors with high therapeutic potential in vivo, in the context of this invention. It should be noted that, as shown in FIG. 2, amplicon vectors also express a GFP/rLuc transgene driven by the HSV-1 IE4/5 promoter.

### Abbreviations:

### Gene Products:

TeNT: light chain of Tetanus neurotoxin
BoNT-X: light chains of Botulinum neurotoxins BoNT-A, -B, -C, -D, -E, or F
BoNT-X-SNARE-Y: fusion proteins in which the light chain of botulinum neurotoxins are fused to the signal and transmembrane peptides of SNARE proteins. More precisely, these transgenes express BoNT-A-syntaxin, BoNT-B-syntaxin or BoNT-C-Vamp2.
GAD67: glutamic acid decarboxylase of 67 kD
NTR: nitroreductase.
Luc: firefly luciferase (fLuc).
Antisense-SNARE: antisense RNA to the SNARE proteins SNAP25, VAMP2 or Syntaxin.
Promoters: Human elongation factor 1 promoter (EF1A), rat Transient Receptor Potential Vanilloide 1 (rTRPV1), human and rat Calcitonin Gene-Related Peptide (hCGRP and rCGRP), rat Acid-Sensing Ion Channel 3 (rASIC3), and human and rat Advillin (hADVL and rADVL) promoters.

FIG. 8. BoNT-A expressed from amplicon vectors cleave the SNARE protein SNAP25 in SH-SY5Y cells.

Human neuroblastoma cells (SH-S5Y5) are infected at an MOI of 01, 1.0, and 10.0 pfu/cell with amplicon vectors expressing transcription units A2-CMV-BoNT-A (LC) or A2-CMV-Luc, driven in both cases by HCMV promoter. The following day, infections were stopped, and cell proteins are analysed by Western blots using antibodies specific for BoNT-A LC and SNAP25. The higher part of the Western blot shows that increasing amounts of BoNT-A LC correspond to increasing MOI, demonstrating that the vectors used do express this protein in the infected cells. The lower part of the blots shows cleavage of SNAP25, the protein from the SNARE complex that is the natural target of BoNT-A, thus producing two fragments. At the lower MOI, mainly the native (not cleaved) form of SNAP25 is observed. At intermediate MOI, both the native and the cleaved form (the slightly lower band) can be seen, while at the higher MOI most of the SNAP25 protein is cleaved, since only the lower fragment of the doublet can be observed. This demonstrates that BoNT-A LC synthesized in SH-S5Y5 cells is able to cleave SNAP25. In contrast, in SH-S5Y5 cells infected with the vector expressing Luc, no cleavage of SNAP25 is observed.

FIG. 9. Light chains of botulin neurotoxins cleave SNARE proteins in infected neurons.

Primary cultures of rat embryonic dorsal root ganglia (DRG) neurons are infected at an MOI of 10 pfu/cell with amplicon vectors expressing transcription units A2-CMV-BoNT-A, A2-CMV-BoNT-B, A2-CMV-BoNT-C, A2-CMV-BoNT-E, and A2-CMV-BoNT-F. Neurons were also infected with amplicon vectors expressing A2-CMV-BoNT-A-syntaxin (STX), A2-CMV-BoNT-B-syntaxin (STX), and A2-CMV-BoNT-C-VAMP2 (V2). Vector expressing A2-CMV-Luc was used as negative control. In all cases, HCMV promoter drove expression of the transcription cassettes. The following day, infections were stopped and cell proteins were analyzed by Westerns blots. As shown in the figure, each BoNT LC synthesized in the neurons cleaved the expected SNARE protein: thus, the light chains of BoNT-A, -C and -E, cleaved SNP25, as evidenced by the decrease in size of this protein, whereas the light chains of BoNT-B, and -F, cleaved VAMP2, which is no more detectable in the blots. In addition, BoNT-C also cleaved Syntaxin (STX), also no more visible in the blots. BoNT-C is the only botulin toxin described to cleave two different SNARE proteins (SNAP25 and STX). The light chains of botulinum toxins fused to the signal and transmembrane peptides of SNARE proteins cleaved the corresponding SNARE proteins exactly as the parental non-fused toxins did. The lane Luc shows the positions of native, non-cleaved, SNARE proteins (arrows). This figure therefore demonstrates that the light chains of botulin toxins (fused or not with fragments of the SNARE proteins) synthesized in sensory neurons upon vector infection, are able to cleave their corresponding target proteins.

FIG. 10. Light chains of botulin toxins inhibit release of neuropeptides in sensory neurons.

Primary cultures of rat embryonic DRG neurons are infected at increasing MOI (from 0.5 to 3 pfu/cell) with amplicon vectors expressing A2-CMV-BoNT-A, A2-CMV-BoNT-B, A2-CMV-BoNT-C, A2-CMV-BoNT-D, A2-CMV-BoNT-E, and A2-CMV-BoNT-F. Neurons were also infected with amplicons expressing A2-CMV-BoNT-A-syntaxin, A2-CMV-BoNT-B-syntaxin, and A2-CMV-BoNT-C-VAMP2. Vector expressing A2-CMV-Luc was used as negative control. Neurons were also infected with vehicle only (mock). The following day, the infected neurons were treated with 75 mM KCl to stimulate release of CGRP, a neuropeptide normally synthesized in DRG neurons. Thirty minutes before and thirty minutes after KCl treatment, 100 microliters aliquots were taken from the culture media and assessed for the presence of CGRP by ELISA (using the CGRP ELISA kit from Spi Bio, ref N^{o} A05482). Results, expressed as linear regression profiles after logarithm conversion, show that all toxins inhibited CGRP release but that they do it with different intensities, with BoNT-F, BoNT-C and BoNT-A being the most effective in this respect. In mock-infected neurons, as well as in neurons infected with the vector expressing Luc, no inhibition of CGRP release was observed. These results clearly indicate that cleavage of SNARE proteins by BoNT LC results in inhibition of neuropeptide release, and that BoNT-F is the most efficient in this respect.

FIG. 11A-11B. GAD67 expressed from amplicon vectors induces synthesis and extracellular release of GABA (gamma amino-butyric acid).

FIG. 11A: Glioblastoma cells (Gli36) were infected at MOI 0.1, 1.0 and 10 pfu/cell with amplicon vectors expressing A2-CMV-GAD67 or A2-CMV-Luc. The following day, infections were stopped and cell proteins were analyzed by Western blots, using antibodies specific for GAD67 and GAPDH (a housekeeping gene used as internal control). Extracts from rat brain were used as positive controls to identify endogenous GAD67. FIG. 11A shows that expression of GAD67 increases with the MOI, demonstrating that vector A2-CMV-GAD67 does express this protein.

FIG. 11B: Primary cultures of rat embryonic DRG neurons were infected at MOI 0.1, 1.0 and 10 pfu/cell with vectors expressing A2-CMV-GAD67 or A2-CMV Luc. The following day infections were stopped and both, intracellular and extracellular, concentrations of GABA were evaluated using Resazurine assay, which is a fluorescence-coupled assay for GABA (the assay is performed as indicated in Ippolito et al., 2014). The upper panel shows that the amount of intracellular GABA increases with the MOI, while the lower panel shows the increase of extracellular GABA. The channel labeled GABA is a positive control for the Resazurine assay. This result clearly shows that expression of GAD67 from the A2-CMV-GAD67 vector increases synthesis of intracellular GABA and its release to the extracellular medium.

FIG. 12A-12B. Nitroreductase (NTR) activates the nitro compound 7′nitrocoumarin and induces cell death in the presence of mitronidazole (MTZ).

FIG. 12A: Human glioblastoma (Gli36) cells were infected with amplicon vectors expressing A2-CMV-NTR or A2-CMV-Luc at an MOI of 1.0 pfu/cell. Two days later, infections were stopped and protein extracts were prepared and used to assess the activation of 7′nitrocoumarin, using a fluorescence-coupled assay (assay performed as in Muller et al. 2015). FIG. 12A shows that only the proteins extracted from cells infected with vector A2-CMV-NTR induced significant activation of 7′nitrocoumarin, demonstrating that functional NTR was expressed in Gli36 cells infected with A2-CMV-NTR.

FIG. 12B: To assess whether expression of NTR induced cell death in the presence of metronidazole (MTZ), Gli36 cells were infected with amplicon vectors A2-CMV-NTR or A2-CMV-Luc at an MOI of 1.0 pfu/cell. The following day cells were incubated with or without MTZ (0.5. mM) for 24 hours. Infections were then stopped and cell viability was assessed using the MTT assay (as indicated by Carmichael et al., 1987). FIG. 12B shows that MTZ significantly increased cell death of infected cells. Mock: non-infected cells.

FIG. 13. Analysis of the selectivity of expression of DRG-selective promoter candidates in autonomic and sensory ganglia from adult rats.

Rat adult sensory ganglia (DRG), autonomic sympathetic ganglia (superior cervical ganglia, SCG), and autonomic parasympathetic ganglia (paracervical ganglia, GPC) were explanted and kept as organotypic cultures. After 3 days, the ganglia were individually infected with vectors expressing A5-TRPV1-Luc, A5-rCGRP-Luc, A5-ASIC3-Luc, or A5-EF1A-Luc, all of them expressing firefly luciferase (fLuc), but driven respectively by the following promoters: rat TRPV1 (rTRPV1), rat CGRP (rCGRP), rat ASIC3 (rASIC3), and EF1a, a non-selective promoter serving as general control. Each ganglion was infected with 10⁶ vector particles. The vectors also express Renilla luciferase (rLuc) driven by a viral promoter (HSV-1 IE4/5). The following day infections were stopped and cells extracts were prepared for luciferase tests using Dual-luciferase reporter assay system from Promega. Results are expressed as ratio of fLuc/rLuc and were normalized as percentage of expression of the EF1a promoter in each of DRG (left), SCG (center) and GPC (right). FIG. 13 shows that some candidate promoters, such as rTRPV1 and rCGRP promoters, express significantly higher levels of fLuc in DRG than in autonomic ganglia, while other promoters, such as rASIC3, do not display preferential activity in DRG. According to these results the rTRPV1 and the rCGRP promoters appear to display selective activity for DRG while rASIC3 does not display such selectivity when expressed from the virus genome.

FIG. 14. shows that an amplicon vector expressing the reporter protein GFP can infect primary cultures of embryonic rat DRG neurons and adult rat DRG explants, and express the transgene GFP within these neurons.

FIG. 15. Intradetrusor inoculation of defective HSV-1 vectors reach dorsal root ganglia (DRG) and express transgenes in sensory neurons innervating the bladder.

Viral vector expressing IE4/5-GFP and HCMV-Luciferase (shown in FIG. 3B) is capable to penetrate and express both transgenic proteins in the bladder afferent neurons following their inoculation into the bladder wall of spinal cord-injured (SCI) rats. DRG neurons expressing both GFP and Luciferase (Luc) are shown in DRG ganglion L6, from which neurons that innervate the bladder extend. However, in the DRG ganglion T13, which does not innervate the bladder, the results are negative. One week post-infection, the animals were sacrificed and transgenic proteins were revealed by IHC using specific antibodies for GFP and Luciferase. These results indicate that following inoculation into the bladder wall, the vectors enter the afferent neurons innervating the bladder and are retrogradely transported through the axons to the cell bodies of the neurons to the L6 ganglia, which lie in the dorsal root ganglia (DRG), from where the viral genome express both transgenic proteins. Vectors are not able to reach or to express in neurons not innervating the bladder (T13).

FIG. 16 shows the high cell selectivity of expression of the viral vector in the dorsal root ganglia (DRG) when Luciferase is driven by the DRG-selective TRPV1 promoter. Luciferase is significantly expressed only in the afferent neurons, and not in the autonomic neurons (sympathetic or parasympathetic). Results were normalized as percentage of luciferase expression relative to that from the vector expressing Luciferase under the control of the strong but not specific HCMV promoter (both vectors are shown in FIG. 3B).

### EXAMPLES

### Example 1: Construction of Defective Recombinant and Amplicon HSV-1 Vectors

### Materials and Methods

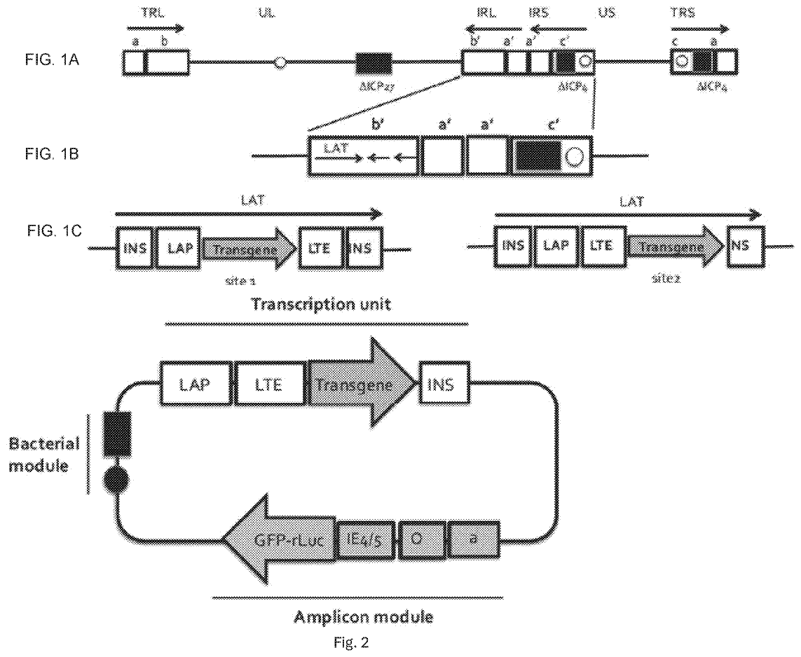

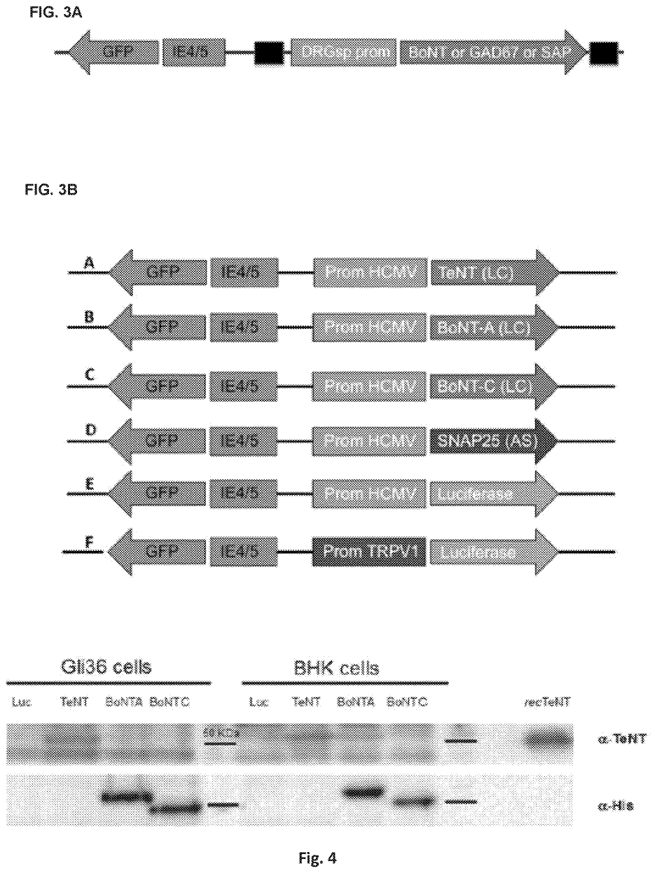

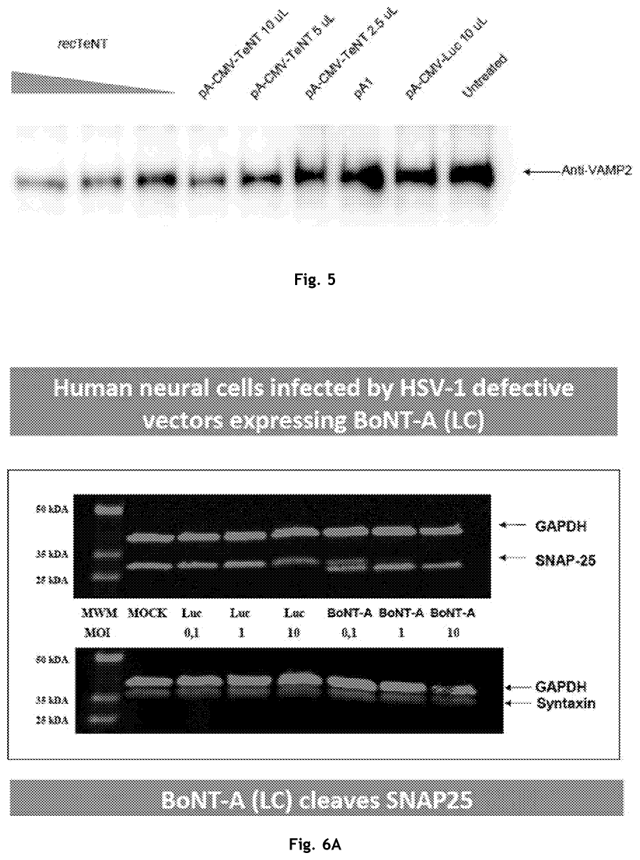

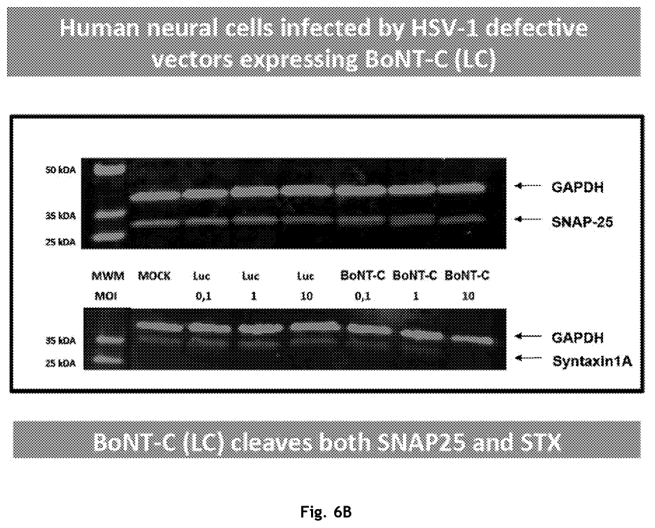

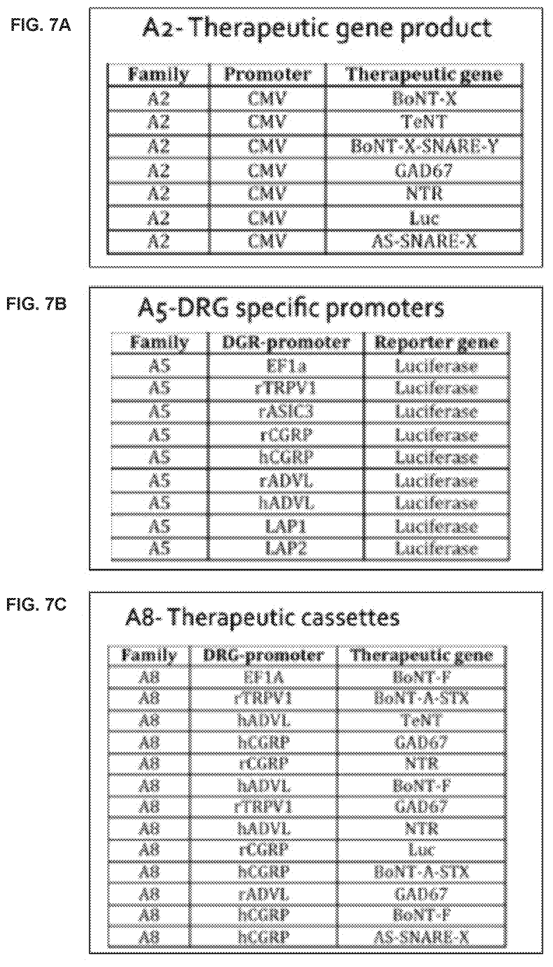

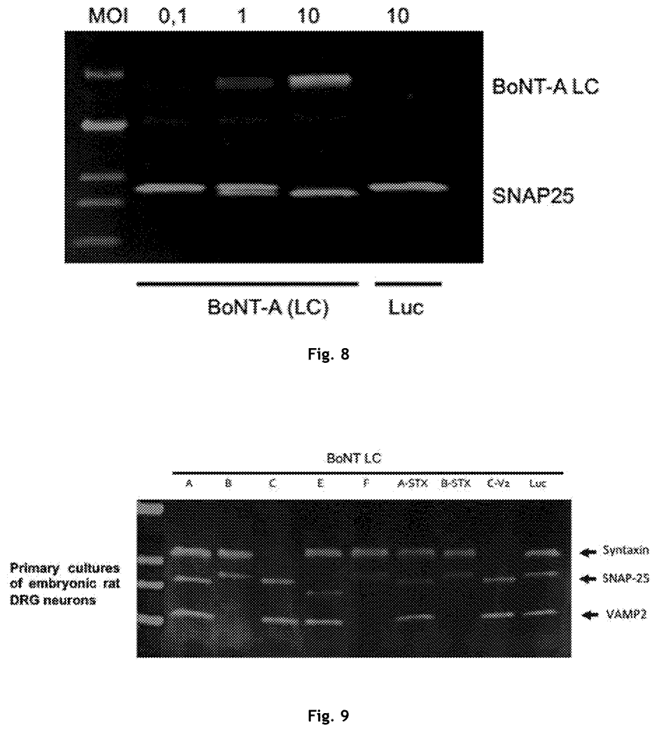

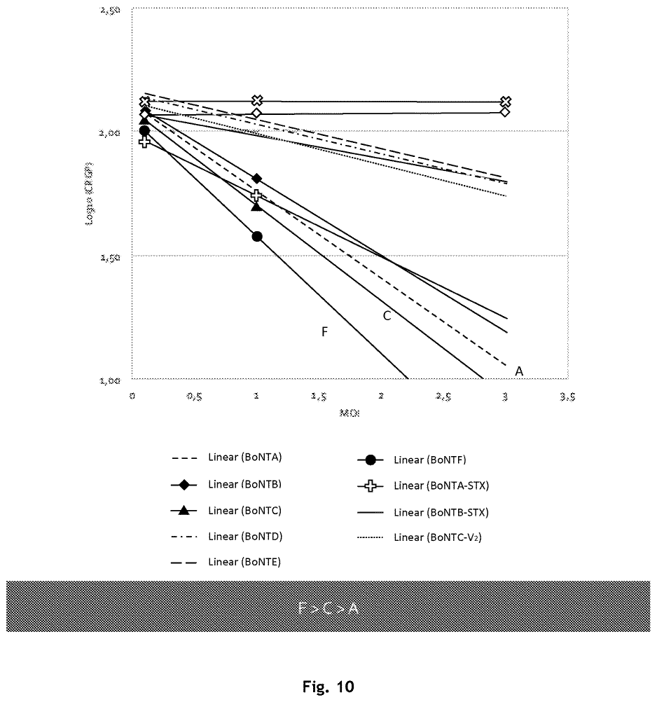

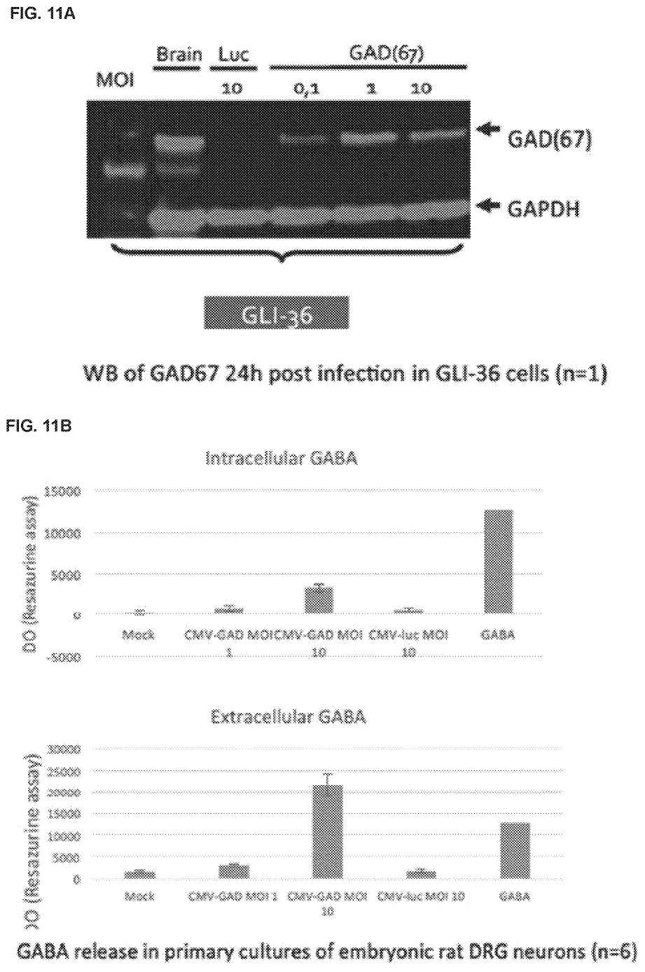

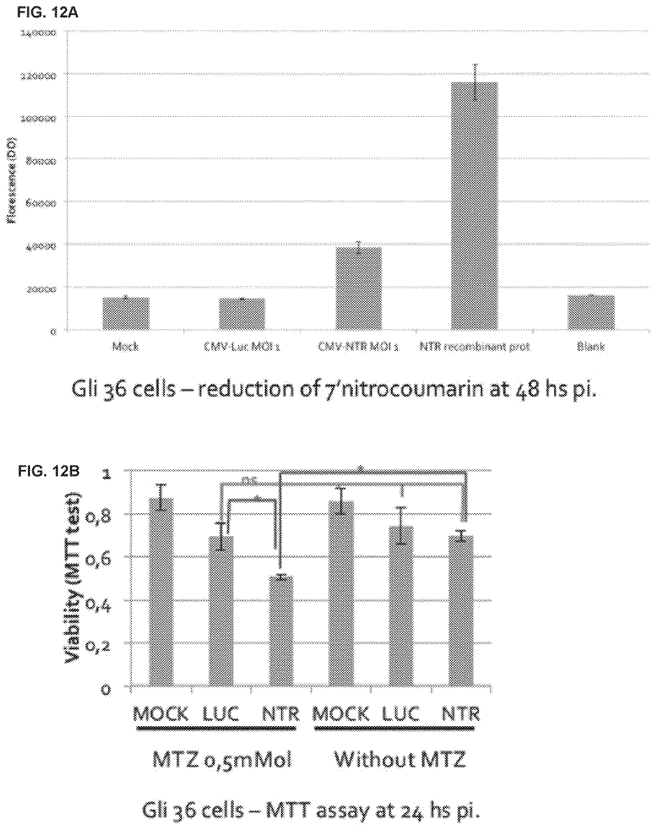

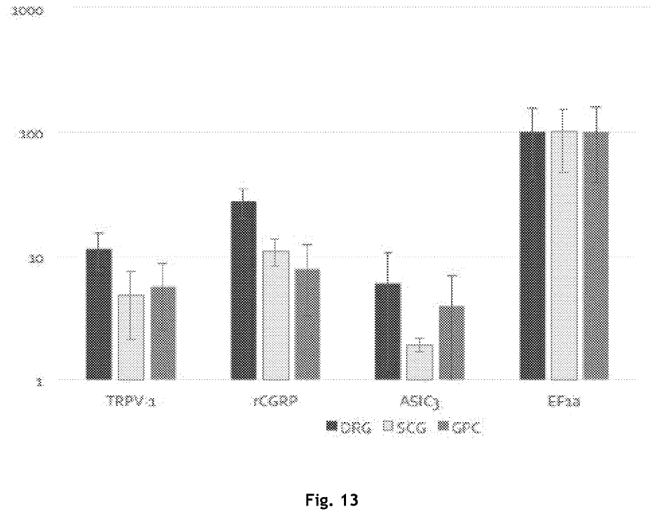

The invention provides set of defective recombinant HSV-1 vectors comprising complete deletions of ICP27 and ICP4 (both copies), and which carries, in addition, the therapeutic transcription cassettes embedded into the LAT locus, either between the LAP and LTE sequences (site 1) or between the LTE and INS sequences (site 2), as shown in FIG. 1A-1C and FIG. 2, to provide long-term expression to said cassette. Some of the transcription cassettes used to generate these vectors are shown in FIG. 3A-3B.

Said transcription cassettes express the light chains (LC) of the Clostridium toxins TeNT (LC), BoNT-A (LC), BoNT B (LC), BoNT-C (LC), BoNT E (LC), BoNT-F (LC), or an antisense RNA (asRNA) directed to the SNARE proteins, VAMP2, SNAP25 and Syntaxin, or fusion SNARE/light-chain toxins, or the human GAD67 protein or a RIP protein such as Saporin S6, or the E. coli NTR nfnB, to inhibit/silence neurotransmission specifically in afferent neurons when, placed under the control of an afferent neuron-specific promoter.

To generate the vectors, we used a full-length HSV-1 genome of strain F cloned into a bacterial artificial chromosome (BAC) such as that described by Tanaka et al, 2003. Gene deletions and gene insertions were introduced by homologous recombination in bacteria and the vectors were then reconstituted by transfection of permissive cell lines as already described (Tanaka et al. 2003). The general structure of these vectors is illustrated in FIG. 1A.

Genome of HSV-1 Amplicon Vectors. FIG. 2.

The invention also provides a set of defective amplicon vectors, which express the same transgenic therapeutic transcriptions cassettes as the recombinant vectors, and listed in FIG. 7A-7C. Sequences conferring long-term expression (LTE and INS) surround the transcription cassettes (FIG. 2). FIG. 2 also shows that in addition to the therapeutic transcription cassettes, amplicon vectors carry a second transcription cassette, expressing a reporter protein (either GFP or the fusion protein GFP/Renilla luciferase) driven in all cases by the HSV-1 IE4/5 promoter.

Amplicon vectors are produced using as helper the defective LaLdeltaJ virus and the complementing cell lines already described by Epstein and collaborators (Zaupa, Revol-Guyot and Epstein, 2003), which expresses the set of proteins required for amplification and packaging of the vector genome.

Transcription Cassettes Carried by Recombinant and Amplicon Vector Genomes. FIG. 7A-7C.

The recombinant and amplicon vectors described in this invention carry and express transgenic transcription cassettes embedded into HSV-1 sequences that confer long-term expression (LAP, LTE, INS), in both types of vectors, as shown in FIGS. 1A-1C and 2. Some examples of the transcription cassettes used in this invention are listed in FIG. 7A-7C.

### Example 2: HSV-1 Amplicon Vectors

The invention also provides a set of defective amplicon vectors, some of these vectors express either reporter proteins (luciferase) or the light chains (LC) of the Clostridium toxins (TeNT (LC), BoNT-A (LC), BoNT B (LC), BoNT-C (LC), BoNT E (LC), BoNT-F (LC)), or an antisense RNA (asRNA) directed to the SNARE proteins, VAMP2, SNAP25 and Syntaxin, or chimeric SNARE/light-chain toxins, or the human GAD67 protein or a RIP protein such as Saporin S6 or a nitroreductase (NTR) protein such as nfnB. The promoters (prom) that drive the expression of these transgenes are either non-specific promoters (HCMV, EF1A), or afferent neuron-specific of promoters (TRPV1, TRPM8, ASIC3, GCRP, ADV1). Additional sequences conferring long-term expression (LTE and DNA insulator sequences) are added to some of these promoters (FIG. 3A). The promoter that governs the expression of the reporter GFP, or the GFP-rLuc fusion protein, present in amplicon vectors, is the viral immediate-early promoter known as HSV-1 IE4/5 promoter. The general structure of some of the amplicon vectors used herein is shown in FIG. 3B.

### Example 3: Expression of BoNT-A, BoNT-C and TeNT. FIG. 4

The expression of BoNT-A (LC), BoNT-C (LC) and TeNT (LC) is performed in Gli36 (a cell line derived from a human glioblastoma) and BHK21 (hamster fibroblast cells) cell lines. Gli36 and BHK21 cells are infected with the amplicon vectors expressing HCMV-Luc, HCMV-BoNT-A (LC), HCMV-BoNT-C (LC), or HCMV-TeNT (LC). The cells were then fixed and the expression of the toxin was demonstrated by Western blot using anti-TeNT antibodies to reveal TeNT (LC) and anti-HIS antibodies to reveal BoNT-A (LC) and BoNT-C (LC). Indeed, there is no efficient anti-BoNT antibodies available, therefore BoNT-A (LC) and BoNT-C (LC) are expressed as a fusion protein with a C-terminal HIS-tag. FIG. 4 shows that the viral vector carrying the genes coding for HCMV-BoNT-A (LC), HCMV-BoNT-C (LC), and HCMV-TeNT (LC) express respectively BoNT-A (LC), BoNT-C (LC) and TeNT (LC) in both Gli36 and BHK21 cells.

### Example 4: In Vitro Proteolytic Activity of the Recombinant Toxin TeNT. FIG. 5

Proteolytic activity of the toxin TeNT (LC) with respect to VAMP2 was evaluated by Westerns blots using anti-VAMP2 antibody. The toxin TeNT (LC) was expressed in Gli36 cells after infection with the viral expression vector expressing HCMV-TeNT (LC). The infection was terminated 2 days later and protein extracts were prepared. These extracts were incubated in a suitable buffer (containing 50 mM Hepes, 400 mM NaCl, 5 mM dithiothreitol and 2 μM ZnSO4) containing the target protein of TeNT, i.e VAMP2. Westerns blots (FIG. 5) were performed using 2.5, 5, and 10 μL of cell extracts. Untreated sample, a sample from cells infected with a vector expressing no transgene (pA-1), and a sample from cells infected with a vector expressing HCMV-Luc (10 μL) were used as a negative control. Varying amounts of recombinant TeNT (recTeNT) were used as a positive control. Results show that the quantity of VAMP2 decreases when the protein extract expressing TeNT (LC) is increased, which demonstrate that the toxin present in the protein extract exhibits a proteolytic activity toward VAMP2.

### Example 5: In Cellulo Proteolytic Activity of the Recombinant Toxins BoNT-A (LC) and BoNT-C (LC). FIG. 6A-6B

The SH-S5Y5 human neuroblastoma cell line was used for their property to spontaneously express SNARE proteins, in order to follow in cellulo SNAP25 and Syntaxin 1a (STX) cleavage following infection by amplicon vectors expressing BoNT-A (LC) or BoNT-C (LC). SNAP25 and STX levels were detected by Western blot assay using anti-SNAP25 or anti-STX antibodies respectively. As negative controls, cells were not infected (Mock) or were infected with the vector expressing HCMV-Luc. Results (FIGS. 6A and 6B show that at 48 hours post-infection (hpi) of SH-S5Y5 cells with vectors expressing the light chains of BoNT-A or BoNT-C, there is respectively cleavage and significant decrease of in cellulo SNAP25 (FIG. 6A) or SNAP25 and STX (FIG. 6B) protein levels relative to cells infected with the control vector expressing Luciferase.

### Example 6: FIG. 8

BoNT-A Expressed from Amplicon Vectors Cleaves the SNARE Protein SNAP25 in SH-SYS5 Cells

This experiment was designed to assess whether vectors expressing the light chain of BoNT-A do express this protein, and to study whether this toxin has the same biological activity that the complete neurotoxin (light chain+heavy chain), i.e., the ability to cleave its target SNARE protein (SNAP25). As shown in FIG. 8, cells infected at increasing multiplicities with amplicon expressing A2-CMV-BoNT-A do express increasing amounts of the toxin. Moreover, when cells are infected at high MOI virtually all SNAP25 is cleaved, clearly demonstrating the functional activity of the light chain of BoNT-A.

### Example 7: FIG. 9

### Light Chains of Botulin Neurotoxins Cleave SNARE Proteins in Infected Neurons.

This experiment was designed to confirm that all BoNT light chains synthesized in vector-infected neurons are able to cleave their natural SNARE target protein in sensory neurons. To this end, primary cultures of rat embryonic DRG neurons were infected at an MOI of 10 with amplicon vectors expressing A2-CMV-BoNT-A, -B, -C, -D, -E and -F, or A2-CMV-Luc as negative control. Infections were stopped the following day and cell extracts were analyzed by Western blots. As shown in FIG. 9, each of the botulinum neurotoxin expressed by the vectors cleaved its natural target SNARE protein. Thus, BoNT-A and -E cleaved SNAP25, BoNT-B, -D and -F cleaved VAMP2, while BoNT-C cleaved both SNAP25 and Syntaxine. This clearly demonstrates that the light chains of all neurotoxins display the same biological activity as the complete neurotoxins (light chain+heavy chain).

### Example 8: FIG. 10

### Light Chains of Botulin Toxins Inhibit Release of Neuropeptides in Sensory Neurons.

This experiment was designed to assess whether the light chains of botulinum neurotoxins induced inhibition of release of neurotransmitters and to evaluate their comparative efficacy in this respect. Primary cultures of rat embryonic DRG neurons were infected at increasing MOI with the vectors as described in FIG. 10. The following day, infected neurons were treated with KCl to stimulated release of neuropeptide CGRP and the extracellular concentrations of CGRP were evaluated by ELISA. As shown in FIG. 10, all neurotoxins induced inhibition of release of CGRP. Moreover, FIG. 6 shows that BoNT-F was the most effective in this respect, followed by BoNT-A and -C.

### Example 9: FIG. 11A-11B

GAD67 Expressed from Amplicon Vectors Induces Synthesis and Extracellular Release of GABA.

The goal of this experiment is to assess whether vectors expressing GAD67 induce synthesis and release of the inhibitory neutransmitter GABA. To this end, glioblastoma cells (Gli36) were infected at increasing MOI with amplicon vectors as described in FIG. 11A and the following day infected cell extracts were analyzed by Western blots, using antibodies specific for GAD67 and GAPDH. FIG. 11A shows that expression of GAD67 increases with the MOI, demonstrating that vector A2-CMV-GAD67 does express this protein. In addition, primary cultures of rat embryonic DRG neurons were infected at different MOIs with the same vectors. The following day infections were stopped and both, intracellular and extracellular, concentrations of GABA were evaluated using Resazurine assay (as indicated in the legend to FIG. 11B. The upper panel of this figure shows that the amount of intracellular GABA increases with the MOI, while the lower panel shows the increase of extracellular GABA, clearly demonstrating that expression of GAD67 from the A2-CMV-GAD67 vector increases synthesis of intracellular GABA and its release to the extracellular medium.

### Example 10: FIG. 12A-12B

### Nitroreductase (NTR) Activates the Nitro Compound 7′Nitrocoumarin and Induces Cell Death in the Presence of Mitronidazole (MTZ).

This experiment was designed to assess whether nitroreductase expressed from amplicon vectors induced cell death in the presence, but not in the absence of metronidazole. There are no available antibodies specific for nitroreductase (NTR). Therefore, to assess that this protein is expressed in A2-CMV-NTR infected cells, we used a functional in vitro test based on the evaluation of reduction of 7′nitrocoumarin (Muller et al., 2015). FIG. 12A shows that amplicon vectors expressing A2-CMV-NTR do activate the nitro compound. Furthermore, FIG. 12B shows that expression of NTR induced significant cell death in the presence of metronidazole (MTZ). This is explained by the fact that NTR can activate MTZ thus transforming this molecule into a cytotoxic drug.

### Example 11: FIG. 13

Analysis of the Selectivity of Expression of DRG-Selective Promoter Candidates in Autonomic and Sensory Ganglia from Adult Rats.

This test was designed to investigate whether afferent neuron-specific promoter candidates, which normally are active only or mainly in afferent neurons, preserve their afferent neurons-specific activity also when they are expressed from the non-replicative HSV-1 vector genome. Rat adult afferent ganglia (DRG), autonomic sympathetic ganglia (SCG), and autonomic parasympathetic ganglia (GPC) were explanted and kept as organotypic cultures. After 3 days, a time required for neurite outgrowth, the ganglia were individually infected with 3×10⁶ vector particles as described in the legend to FIG. 13. These vectors express firefly luciferase (fLuc) driven by the following promoters: rat TRPV1 (rTRPV1), rat CGRP (rCGRP), rat ASIC3 (rASIC3), all of which are considered as afferent-neuron specific promoters, and EF1a, a non-selective promoter serving as general control. In addition to fLuc, these vectors also express Renilla luciferase (rLuc) driven by a viral promoter (HSV-1 IE4/5). The following day infections were stopped and cells extracts were prepared for luciferase tests. Results are expressed as the ratio of fLuc/rLuc and as percentage of luciferase activity driven by EF1a. FIG. 13 shows that rTRPV1 and rCGRP express firefly luciferase activity preferentially in DRG and can thus be considered as DRG-specific even when they express from the vector genome. In contrast, rASIC3 does not display such preferential expression in the DRG demonstrating that this promoter does not preserve its selectivity when expressed from the vector genome. Therefore, this example shows that some DRG-specific promoter candidates, such as the rTRPV1 and rCGRP promoters, do preserve their selectivity for DRG while other promoter candidates, such as rASIC3, although considered a DRG-specific promoter when it expresses from the cellular chromosomes, does not preserve this specificity when expressed from the vector genome. Therefore, the behavior of any particular DRG-specific promoter candidate cannot be predicted and should be experimentally assessed.

### Example 12: Infection and Expression of the Recombinant Protein in Cell Cultures

Primary rat neuronal cultures from embryonic DRG and organotypic cultures of adult rat DRG explants were infected with and amplicon vector expressing GFP driven by the HSV-1 immediate early IE4/5 promoter. Results show that the viral expression vector infected and expressed the transgene (GFP) both in primary rat sensory neuronal cultures and in adult rat ganglion (DRG) explants (FIG. 14).

### Example 13: In Vivo Expression of Recombinant Proteins in Neurons

Spinal cord injured (SCI) rats were infected by the amplicon vector HCMV-Luc, which simultaneously expresses GFP and Luc reporter proteins. One week post-infection, the animals were sacrificed and transgenic proteins expressions were revealed by IHC. As indicated by the IHC, when inoculated into the bladder the amplicon vector is entering the afferent neurons innervating the bladder, and is then retrogradely transported through the axons to the cell bodies of the neurons, which lie in the dorsal ganglia (DRG), and where the viral genome express both transgenic protein. Results indicate that amplicon vectors HCMV-Luc are thus capable to penetrate and specifically express transgenic proteins into the bladder afferent neurons following their inoculation into the bladder wall (FIG. 15). Moreover, neurons expressing GFP and Luc are observed only in the ganglion from which neurons that innervate the bladder extend (the L6 ganglion). In contrast, in the ganglion T13, which does not innervate the bladder, no transgene expression could be observed (data not shown).

### Example 14: Cell Specificity Expression of the Viral Expression Vector

The amplicon vectors TRPV1-Luc, expressing luciferase under control of the promoter TRPV1 (promoter active selectively in afferent neurons) and HCMV-Luc, expressing luciferase under the control of the non-selective HCMV promoter, were used to infect sensory or autonomic ganglia (both sympathetic and parasympathetic ganglia). Results show that expression of the luciferase under TRPV1 promoter is specifically expressed in the afferent neurons of the sensory ganglia (Dorsal Root Ganglia, DRG), and not in the autonomic neurons (sympathetic or parasympathetic) (FIG. 16). Results are expressed as percentage of expression driven by the non-selective HCMV promoter, which is equally high in all types of ganglia.

While this invention has been particularly shown and described with references to preferred embodiments thereof, it will be understood by those skilled in the art that various changes in form and details may be made therein without departing from the scope of the invention encompassed by the appended claims.

### BIBLIOGRAPHIC REFERENCES

Amelio A L, McAnany P K and Bloom D C. A chromatin insulator-like element in the herpes simplex virus type 1 latency-associated transcription region binds CCCTC-binding factor and displays enhancer-blocking and silencing activities. Journal of Virology 80: 2358-2368 (2006).
Berthomme H, Lokensgard J, Yang L, Margolis T, and Feldman L T. Evidence for bidirectional element located downstream from the herpes virus simplex type 1 latency-associated promoter that increases its activity during latency Journal of Virology 74:3613-3622 (2000).
Berthomme H, Thomas J, Texier P, Epstein A and Feldman L T. Enhancer and long-term expression functions of herpes simplex virus type 1 latency-associated promoter are both located in the same region. Journal of Virology 75: 4386-4393 (2001).
Brindley G S, Polkey C E, Rushton D N, and Cardozo L. Sacral anterior root stimulators for bladder control in paraplegia: the first 50 cases. J Neurol Neurosurg Psychiatry 49: 1104-1114 (1986).
De Groat W C. Spinal cord projections and neuropeptides in visceral afferent neurons. Prog Brain Res 67:165-187 (1986).
De Groat W C and Yoshimura N. Mechanisms underlying the recovery of lower urinary tract function following spinal cord injury. Prog Brain Res 152: 59-84 (2006).
Epstein A L. HSV-1-derived amplicon vectors: recent technological improvements and remaining difficulties—A review. Mem Inst Oswaldo Cruz 104: 339-410 (2009).
Fowler C J, Griffiths D and de Groat W C. The neural control of micturition. Nat Rev Neurosci 9: 453-466 (2008).
Furuta Y, Takasu T, Fukuda S, Inuyama Y, Sato K C, Nagashima K. Latent herpes simplex virus type 1 in human vestibular ganglia. Acta Otolaryngol Suppl. 1993; 503:85-9.
Habermann E, and Dreyer F. Clostridial neurotoxins: handling and action at the cellular and molecular level. Curr. Top. Microbiol. Immunol. 129: 93-179 (1986).
Lokensgard J R, Berthomme H and Feldman L T. The latency-associated promoter of herpes simplex virus type 1 requires a region downstream of the transcription start site for long-term expression during latency. Journal of Virology 71: 6714-6719 (1997).
Marconi P, Manservigi R and Epstein A L. HSV-1-derived helper-independent defective vectors, replicating vectors and amplicon vectors, for the treatment of brain diseases. Current opinion in drug discovery and development 13: 169-183 (2010).
McCart J A1, Wang Z H, Xu H, Hu Y, Park B, Alexander H R, Bartlett D L. Development of a melanoma-specific adenovirus. Mol Ther. 2002 October; 6(4):471-80.
Morrison J F, Birder L, Craggs M, de Groat W C, Downie J W, Drake M, Fowler C J, Thor K B. Neural control. In: Abrams P, Cardozo L, Khoury S, Wein A, editors. Incontinence. Plymouth: Health, 363-422 (2005).
Perng G, Ghiasi H, Slavina S M, Nesburn A B, and Wechsler S. The spontaneous reactivation function of the herpes simplex virus type 1 LAT gene resides completely within the first 1.5 kilobases of the 8.3-kilobase primary transcript. Journal of Virology 70: 976-984 (1996).
Ren J, Chew D J, Biers S, Thiruchelvam N. Electrical nerve stimulation to promote micturition in spinal cord injury patients: A review of current attempts. Neurourol Urodyn. doi: 10.1002/nau.22730. [Epub ahead of print](2105).
Sugiyama H. Clostridium botulinum neurotoxin. Microbiol. Rev. 44, pp. 419-448 (1980).
Tanaka M, Kagawa H, Yamanashi Y, Sata T, and Kawaguchi Y. Construction of an excisable bacterial artificial chromosome containing a full-length infectious clone of herpes simplex virus type 1: viruses reconstituted from the clone exhibit wild-type properties in vitro and in vivo. Journal of Virology 77: 1382-1391 (2003).
Warren K G, Brown S M, Wroblewska Z, Gilden D, Koprowski H, Subak-Sharpe J. Isolation of latent herpes simplex virus from the superior cervical and vagus ganglions of human beings. N Engl J Med. 1978 May 11; 298(19):1068-9.
Zaupa C, Revol-Guyot V and Epstein A L. Improved packaging system for generation of high-level noncytotoxic HSV-1 amplicon vectors using Cre-loxP site-specific recombination to delete the packaging signals of defective helper genomes. Human Gene Therapy 14: 1049-1063 (2003).

1-19. (canceled)

20. A defective recombinant herpes simplex virus 1 (HSV-1) viral expression vector comprising:
a) a deletion of one or more HSV-1 essential proteins;
b) at least one transcription cassette comprising a nucleotide sequence encoding a transgene operably linked to a promoter active selectively in afferent neurons, wherein said transgene encodes for a wild-type or a modified bacterial neurotoxin light chain of Clostridium tetani or botulinum or an active fragment thereof; and
c) at least one sequence conferring long-term expression, wherein said long-term expression sequence is an LTE and a DNA insulator from the HSV-1 genome and wherein said transcription cassette is placed between the LTE and the DNA insulator.

21. The viral expression vector according to claim 20, wherein the one or more HSV-1 essential proteins are selected from Infected Cell Protein 4 (ICP4) and ICP27.

22. The viral expression vector according to claim 20, wherein one or both copies of HSV-1 essential protein ICP4 are deleted.

23. The viral expression vector according to claim 22, both copies of ICP4 are deleted.

24. The viral expression vector according to claim 20, wherein the HSV-1 essential protein ICP27 is deleted.

25. The viral expression vector according to claim 22, further comprising the deletion of HSV-1 essential protein ICP27.

26. The viral expression vector according to claim 20, further comprising the deletion of one or more HSV-1 non-essential proteins.

27. The viral expression vector according to claim 26, wherein the HSV non-essential proteins are selected from ICP34.5, UL55, UL56, and UL41.

28. The viral expression vector according to claim 20, wherein said promoter is selected from the group consisting of a promoter of Calcitonin Gene Related Peptide (CGRP), the promoter of Substance P, and a promoter of the TRP gene family.

29. The viral expression vector according to claim 20, wherein said light chain of said bacterial neurotoxin is selected from a light chain of the botulinum neurotoxin A (BoNT-A), a light chain of the botulinum neurotoxin B (BoNT-B), a light chain of the botulinum neurotoxin C1 (BoNT-C1), a light chain of the botulinum neurotoxin E3 (BoNT-E3), a light chain of the botulinum neurotoxin F1 (BoNT-F1), or a light chain of the tetanic neurotoxin (TeNT).

30. The viral expression vector according to claim 29, wherein said light chain of said bacterial neurotoxin is a light chain of the botulinum neurotoxin F1 (BoNT-F1).

31. A pharmaceutical composition comprising at least one viral expression vector according to claim 20.

32. A kit comprising at least one viral expression vector according to claim 20, and an electrical stimulation system comprising electrodes to be implanted on the sacral anterior roots, to apply intermittent stimulation pulse trains in order to achieve a sustained detrusor muscle contraction with intervals of urethral sphincter relaxation allowing urine to flow.

33. The kit according to claim 32, wherein the sacral anterior roots are S2, S3, and/or S4.
